(19) United States
(12) Patent Application Publication
(10) Pub. No.: US 2024/0122845 A1
(43) Pub. Date: Apr. 18, 2024

(54) ADMINISTRATION REGIMENS FOR MICRONEEDLE PATCH DEVICES FOR DELIVERY OF IMMUNOGENIC COMPOSITIONS

(71) Applicants: Artificial Cell Technologies, Inc., New Haven, CT (US); Georgia Tech Research Corporation, Atlanta, GA (US)

(72) Inventors: Thomas J. Powell, Madison, CT (US); Matthew Joseph Mistilis, Atlanta, GA (US); Mark Prausnitz, Atlanta, GA (US)

(21) Appl. No.: 18/470,587

(22) Filed: Sep. 20, 2023

Related U.S. Application Data
(60) Provisional application No. 63/408,269, filed on Sep. 20, 2022.
(60) Provisional application No. 63/501,550, filed on May 11, 2023.

(51) Int. Cl. A61K 9/00; A61K 39/015; A61K 39/12; A61K 47/38; A61M 37/00; A61P 31/14; A61P 33/06

(52) U.S. Cl. CPC A61K 9/0021; A61K 39/015; A61K 39/12; A61K 47/38; A61M 37/0015; A61P 31/14; A61P 33/06; A61K 2039/627; A61M 2037/0023; A61M 2037/0061; A61M 2202/30

(57) ABSTRACT

Described herein are methods of eliciting an immune response in human subject in need thereof, the methods including transdermally delivering to the human subject a composition comprising particles, the composition contained in a housing of a microneedle patch device, wherein transdermally delivering includes transdermally delivering an initial dose; transdermally delivering a booster dose within 4-6 weeks of the initial dose; and transdermally delivering a subsequent dose at 1 year or more after delivering the booster dose, wherein no dose is given between the booster dose and the subsequent dose. The composition includes particles including an antigenic multilayer film.

### CROSS-REFERENCE TO RELATED APPLICATIONS

This application claims priority to U.S. Provisional Application Ser. No. 63/408,269 filed on Sep. 20, 2022, and 63/501,550, filed May 11, 2023, which are incorporated herein by reference in their entirety.

### SEQUENCE LISTING

The Instant Application contains a Sequence Listing which has been submitted electronically in XML format and is hereby incorporated by reference in its entirety. Said XML copy, created on Sep. 18, 2023 is named SEQ LIST—107650_001 and is 20.3 KB (20,876 bytes) in size. The Sequence Listing does not go beyond the disclosure in the application as filed.

### FIELD OF THE DISCLOSURE

The present disclosure relates to administration regimens for microneedle patch devices which deliver immunogenic compositions.

### BACKGROUND

As described in U.S. Pat. No. 7,615,530, electrostatic layer-by-layer multilayer films provide a platform for immunogenic compositions for use as vaccines, for example. In an electrostatic layer-by-layer multilayer film, deposition of oppositely charged polyelectrolytes onto a surface, such as a particle, provides a stable multilayer structure. Polypeptide epitopes can be incorporated into a charged polyelectrolyte such as a polypeptide, allowing for incorporation of a polypeptide epitope into the film. The films containing the epitopes can be used to elicit an immune response and provide protection against a target, such as a pathogen.

While the compositions disclosed in U.S. Pat. No. 7,615,530 are suitable for their intended purpose, it would be advantageous to provide alternate delivery systems that provide favorable administration regimens.

### SUMMARY

In an aspect a method of eliciting an immune response in human subject in need thereof comprises transdermally delivering to the human subject a composition comprising immunogenic particles by a microneedle patch device, wherein transdermally delivering comprises: transdermally delivering an initial dose by the microneedle patch device; optionally transdermally delivering a booster dose by the microneedle patch device within 3-12 weeks of the initial dose; and transdermally delivering a subsequent dose by the microneedle patch device at 1 year or more after delivering the initial or booster dose, wherein no dose is given between the initial or booster dose and the subsequent dose. The composition comprising immunogenic particles comprises a multilayer film, the multilayer film comprising two or more layers of charged polyelectrolytes, wherein adjacent layers comprise oppositely charged polyelectrolytes, one of the charged polyelectrolyte layers in the multilayer film comprises an antigenic polyelectrolyte comprising a peptide epitope covalently linked to the antigenic polyelectrolyte, wherein the polyelectrolytes that are not the antigenic polyelectrolyte comprise a polycationic material or a polyanionic material having a molecular weight of greater than 1,000 and at least 5 charges per molecule, and wherein the multilayer film is deposited on a core particle or forms a hollow particle to provide the composition. The microneedle patch device comprises a substrate comprising an array of microneedles extending therefrom, wherein the microneedles comprise the composition comprising immunogenic particles.

### BRIEF DESCRIPTION OF THE DRAWINGS

FIG. 1 shows the induction of in vivo CTL activity by LbL-MP recovered from microneedle patch tips. Individual mice were immunized as shown, then challenged on day 7 with labeled target cells. The next day, host spleens were harvested, and cells were analyzed by flow cytometry to detect survival of the differentially labeled target cells. The graph shows % specific (mean±SD of 2 or 3 mice per group).

FIG. 2 shows induction of in vivo CTL activity by microneedle patches formulated with ACT-1232. Individual mice were immunized as shown, then challenged on day 7 with labeled target cells. The next day, host spleens were harvested, and cells were analyzed by flow cytometry to detect survival of the differentially labeled target cells. The graph shows % specific (mean±SD of 3 mice per group).

FIGS. 3A and B show IL-5 and IFNγ T-cell ELISPOTs in mice immunized with RSV-GM2 microparticles, patches, or tips recovered from patches. Mice were immunized on days 0 and 28. On day 8 (3A) and again on day 35 (3B), three mice/group were sacrificed, and spleen cells were harvested and restimulated in vitro with RSV-M2 and G peptides in IL-5 and IFNγ ELISPOT. The data depict the mean±SD spots per 106 spleen cells of 3 mice/groups.

FIGS. 4A-D show antibody responses elicited by microneedle patches formulated with ACT-1232. BALB/c mice were immunized with the indicated treatments on day 0 and 28. On day 35, sera were tested in ELISA against RSV-G peptide. (4A) RSV-G peptide-specific IgG. Data depict the mean±SD of 13 mice per group. (4B) Results show individual mice (circles) and group averages (bars) at 1:50 serum dilution. (4C) Isotype distribution of RSV-G-peptide specific was measured at 1:50 dilution of serum. Data depict the mean±SD of 13 mice per group. (4D) The IgG1:IgG2a ratio was calculated by dividing OD values for IgG1 by OD values for IgG2a.

FIG. 5 shows viral burden in lungs. Mice were immunized on days 0 and 28 and challenged with RSV on day 50. Five days post-challenge, mice were sacrificed, and lung virus burden was measured by standard plaque assay on Vero cells. Results show individual mice (circles) and group means (bars). Insets show % reduction in viral burden (group average vs naïve group average), P-value (vs naïve group), and the number of mice completely protected in each group (no plaques detected).

FIG. 6 shows antibody responses elicited by microneedle patches formulated with ACT-1230 and -1231. C57BL/6J mice were immunized with the indicated treatments on day 0. On day 21, sera were tested in ELISA against T1B peptide. Results show individual mice (circles) and group averages (bars) at 1:50 serum dilution.

FIGS. 7A and B show antibody responses elicited by microneedle patches formulated with ACT-1230 and -1231. C57BL/6J mice were immunized with the indicated treatments on day 0 and 30. On day 37, sera were tested in ELISA against T1B peptide. (7A) T1B peptide-specific IgG. Data depict the mean±SD of 10 mice per group. (7B) Isotype distribution of T1B-peptide specific was measured at 1:50 dilution of serum. Data depict the mean±SD of 10 mice per group.

FIG. 8 shows a T-cell ELISPOTs in mice immunized with malaria T1BT* or Pam3Cys.T1BT* microparticles or microneedle patches. Mice were immunized on days 0 and 30. On day 37, three mice per group were sacrificed and spleen cells were harvested and restimulated in vitro with T1B peptide in IL-5 and IFNγ ELISPOT. Data depict the mean±SD spots per 106 spleen cells of 3 mice per group.

FIG. 9 shows the persistence of antibody response elicited by microneedle patches formulated with ACT-1230 and -1231. C57BL/6J mice were immunized with the indicated treatments on day 0 and 30. Sera collected 7 and 90 days post-boost were tested in ELISA against T1B peptide. Results show individual mice (circles) and group averages (bars) at 1:250 serum dilution.

FIG. 10 shows the persistence of antibody response elicited by microneedle patches formulated with ACT-1230 and -1231. C57BL/6J mice were immunized with the indicated treatments on days 0 and 30. Sera collected 7, 90 and 180 days post-boost were tested in ELISA against T1B peptide. Results show individual mice (circles) and group averages (bars) at 1:250 serum dilution.

FIG. 11 shows the persistence of antibody responses elicited by microneedle patches formulated with ACT-1230 and -1231. C57BL/6J mice were immunized with the indicated treatments (vertical labels on left side of graphic) on days 0 and 30. Sera were collected 7, 180 and 540 days post-boost (horizontal labels on top of graphic) and tested in ELISA against T1B peptide. Results show serial dilutions of sera from individual mice. Individual mouse identification numbers for each row of graphs are shown in the center graph in that row.

FIGS. 12A-C are a schematic of an embodiment of a microneedle patch delivery device.

FIGS. 13A-C show a comparison of vaccine-loaded LbL-MPs applied via IM and microneedle patch. 13A shows T1B-specific IgG response versus vaccine dose. 13B shows prime versus prime-boost cellular immune responses, ****p<0.0001. 13C shows T1B-specific IgG response on days 7 and 180.

The above-described and other features will be appreciated and understood by those skilled in the art from the following detailed description, drawings, and appended claims.

### DETAILED DESCRIPTION

Disclosed herein are microneedle patch delivery devices for the administration of particles comprising an antigenic multilayer film, and methods of administration/eliciting immune responses with the microneedle patch devices. Unexpectedly, once a primary dose and optionally a booster dose are administered, the administration of the particles using the microneedle patch devices is not repeated for more than 1, 2 or even 3 years. This is a significant advantage over the RTS,S malaria vaccine which is administered as 3 monthly doses starting at age 5 months followed by a fourth dose 15-18 months later. The microneedle patch delivery devices, in addition to providing a long-lived antibody response comparable to that elicited by parenteral immunization, favor a TH1 phenotype over a TH2 phenotype.

In an aspect, a method of eliciting an immune response in a human subject in need thereof comprises transdermally delivering to the human subject a composition comprising immunogenic particles by a microneedle patch device, wherein transdermally delivering comprises: transdermally delivering an initial dose by the microneedle patch device; optionally transdermally delivering a booster dose by the microneedle patch device within 3-12 weeks of the initial dose; and transdermally delivering a subsequent dose by the microneedle patch device at 1 year or more after delivering the initial or the booster dose, wherein no dose is given between the initial or booster dose and the subsequent dose. The composition comprising immunogenic particles comprises a multilayer film, the multilayer film comprising two or more layers of charged polyelectrolytes, wherein adjacent layers comprise oppositely charged polyelectrolytes, one of the charged polyelectrolyte layers in the multilayer film comprises an antigenic polyelectrolyte comprising a peptide epitope covalently linked to the antigenic polyelectrolyte, wherein the polyelectrolytes that are not the antigenic polyelectrolyte comprise a polycationic material or a polyanionic material having a molecular weight of greater than 1,000 and at least 5 charges per molecule, and wherein the multilayer film is deposited on a core particle or forms a hollow particle to provide the composition. The microneedle patch device comprises a substrate comprising an array of microneedles, e.g., bioerodible or biodegradable microneedles, extending therefrom, wherein the microneedles comprise the composition comprising immunogenic particles.

In an aspect, the subsequent dose is administered at 18 months, 2 years, 3 years or more after delivering the initial or the booster dose, wherein no dose is given between the initial or booster dose and the subsequent dose.

In an aspect, microneedles and a microneedle delivery device are described in US20210196937, US20200238065, and US20180133447 incorporated herein by reference for their disclosure of microneedles and microneedle patch devices.

In an aspect, a microneedle patch delivery device for delivering immunogenic particles with an array of microneedles, e.g., separable microneedles, is provided. In an embodiment, the drug delivery device with microneedles includes a substrate having a microneedle side and an opposing back side, an array of microneedles extending from the microneedle side of the substrate, wherein the microneedles comprise the immunogenic microparticles. A supporting layer can be arranged, e.g., adhered, on the opposing back side of the substrate. The substrate may also include at least one feature configured to separate the array of microneedles from the substrate upon application of a force to the substrate sufficient to at least partially penetrate a tissue surface with the array of microneedles.

In another aspect, the drug delivery device having microneedles, e.g., separable microneedles, includes a housing for the substrate and the supporting layer, the housing having a depressible portion, wherein the substrate and the supporting layer are movably mounted within the housing, wherein the depressible portion is configured to apply or activate upon depression a shearing force to at least one of the supporting layer and substrate effective to separate the array of microneedles from the substrate. The shearing force, in embodiments, is a rotational or linear/lateral shearing force. The drug delivery device may also include an apparatus that applies a shearing force upon depression of the depressible portion.

In another aspect, a method of inserting microneedles into a biological tissue for administering a drug into the biological tissue is provided. In embodiments, the methods include positioning a microneedle patch device on the biological tissue surface, the microneedle patch delivery device comprising an array of microneedles, which comprise the immunogenic microparticles, extending from a substrate, and applying a force to the device effective to (i) penetrate the tissue surface with the array of microneedles, and (ii) optionally separate the array of microneedles from the substrate. The positioning and applying steps may individually or both be performed manually. In one embodiment, penetration of the tissue surface and separation of the array of microneedles from the substrate occur substantially simultaneously. For example, a user can manually apply the device against a person's skin, and simply depress a button or other portion of the device, or twist the device, to both insert the microneedles into the skin and separate the microneedles from the device, in a simple and quick motion. This advantageously simplifies the administration process and avoids the need to have some external device portion remain on the skin surface for a prolonged period, e.g., during drug release or while waiting for a dissolution-driven separation to occur.

In another aspect, a drug delivery device is provided that is capable of controlling the rate and/or direction of immunogenic particle release. In one embodiment, the microneedle patch device includes an array of microneedles which comprise immunogenic particles and which extend from a base, and a system for triggering, after the microneedles are inserted at least partially into a biological tissue, a change in rate of release of the immunogenic particles from the microneedles and into the biological tissue. In another embodiment, the microneedle patch device includes a substrate having a microneedle side and an opposing back side, an array of microneedles extending from the microneedle side of the substrate, wherein the microneedles comprise immunogenic particles, a supporting layer arranged on the opposing back side of the substrate, and a barrier configured to permit (i) discrete periods of immunogenic particle release upon or after implantation, (ii) control of the region of the microneedles from which the immunogenic particles are released, or (iii) a combination thereof. In a further embodiment, the microneedle patch delivery devices include a barrier that is capable of controlling immunogenic particle release rate and/or location of immunogenic particle release.

In an aspect, the separation of the microneedles from the substrate occurs during application of the input force by a user. In contrast, a conventional system describes separation to occur based on a dissolution process that occurs after microneedle insertion and after no more force is applied to the microneedle device. In such conventional cases, at some later time (e.g., several minutes or hours), the microneedles (or a portion of the microneedles) get wet and soft and may form a gel and partially dissolve such that the substrate can be removed from the tissue, and the microneedles stay behind in the tissue. Again in contrast, with the devices and methods described herein, separation of the microneedles advantageously is not facilitated (at all or substantially) by interaction of the microneedles with water in the tissue or imbibing water or dissolving or any other such process.

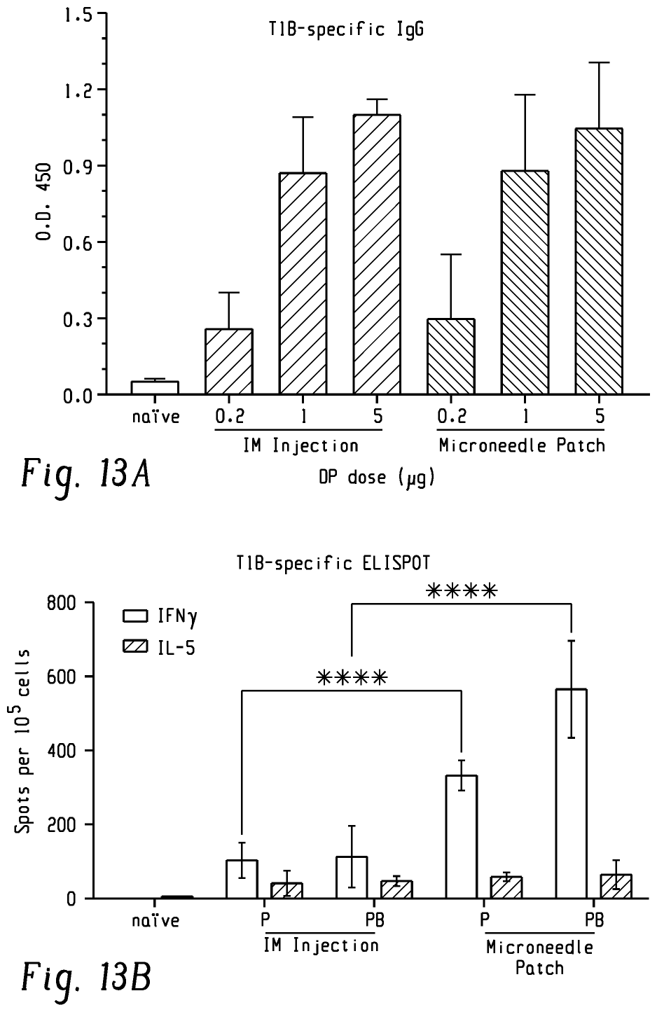

One embodiment of a microneedle patch delivery device is depicted at FIGS. 12A-C. The drug delivery device 100 includes a supporting layer 110 and a substrate 120 from which an array of microneedles 130 extends (FIG. 12A). The microneedles 130 of the drug delivery device 100 penetrate a tissue surface 150 (FIG. 12B), which results in fractured microneedles 160 (FIG. 12C), upon the application of a force. The microneedles of FIG. 12A include a predefined fracture region 140, but the presence of the predefined fracture region 140 is not required.

The microneedle arrays include two or more microneedles which extend from a surface of a base substrate. The phrase “base substrate” and the term “substrate” are used interchangeably herein. Each microneedle has a proximal end attached to the base substrate directly, or indirectly such as via one or more predefined fracture regions, and a distal tip end which is sharp and effective to penetrate biological tissue. The microneedle may have tapered sidewalls between the proximal and distal ends.

The length of a microneedle may be between about 50 μm and 2 mm. In most cases they are between about 200 μm and 1200 μm, and ideally between about 500 μm and 1000 μm. The volume of a microneedle can be between about 1 nl and 100 nl. In most cases, it is between about 5 nl and 20 nl.

In one embodiment, the array of microneedles includes from 10 to 1000 microneedles.

In a preferred embodiment, the microneedles are solid microneedles that include immunogenic particles, which are delivered in vivo following insertion of the microneedle into a biological tissue, e.g., into the skin of a patient. For example, the immunogenic particles may be mixed into a water soluble matrix material forming a solid microneedle. The immunogenic particles may be provided in a formulation which is bioerodible. As used herein, the term “bioerodible” means that the structure/material degrades in vivo by dissolution, enzymatic bond cleavage, hydrolysis, erosion, resorption, or a combination thereof. In a preferred embodiment, the immunogenic particles and a matrix material in which the immunogenic particles are dispersed form the structure of the microneedle. In a preferred embodiment, the matrix material of the bioerodible microneedle is water soluble, such that the entire microneedle dissolves in vivo. In another embodiment, the matrix material of the bioerodible microneedle is biodegradable, such that the microneedles are not soluble in the form originally inserted into the biological tissue, but undergo a chemical change in the body (e.g., break chemical bonds of a polymer) that renders the products of the chemical change (e.g., monomers or oligomers of the polymer) water soluble or otherwise clearable from the body.

The immunogenic particles may be inside and/or on the surface of the microneedles, inside and/or on the substrate, or a combination thereof. The immunogenic particles may be dispersed in a particular region of the microneedles, disposed in one or more reservoirs within the microneedles, disposed in an area of high concentration, or a combination thereof.

In an aspect, a matrix material forms the bulk of the microneedle and substrate. It typically includes a biocompatible polymeric material, alone or in combination with other materials. In embodiments, the matrix material, at least of the microneedles, is water soluble. Exemplary matrix materials include one or a combination of polyvinyl alcohol, dextran, carboxymethylcellulose, maltodextrin, sucrose, trehalose, and other sugars. As used herein, the terms “matrix material” and “excipient” are used interchangeably when referring to any excipients that are not volatilized during drying and formation of the microneedles and substrate.

A solution including the matrix material and the immunogenic particles can be filled into a mold to provide the microneedles. The fluid solution used in the mold filling processes described herein may include any of a variety of excipients. The excipients may consist of those that are widely used in pharmaceutical formulations or ones that are novel. In a preferred embodiment, the excipients are ones in FDA-approved drug products. Exemplary excipients include stabilizers, buffers, bulking agents or fillers, adjuvants, surfactants, disintegrants, antioxidants, solubilizers, lyo-protectants, antimicrobials, antiadherents, colors, lubricants, viscosity enhancer, glidants, preservatives, materials for prolonging or controlling delivery (e.g., biodegradable polymers, gels, depot forming materials, and others). Also, a single excipient may perform more than one formulation role. For example, a sugar may be used as a stabilizer and a bulking agent, or a buffer may be used to both buffer pH and protect the active from oxidation. Some examples of excipients include, but are not limited to, lactose, sucrose, glucose, mannitol, sorbitol, trehalose, fructose, galactose, dextrose, xylitol, maltitol, raffinose, dextran, cyclodextrin, collagen, glycine, histidine, calcium carbonate, magnesium stearate, serum albumin (human and/or animal sources), gelatin, chitosan, DNA, hyaluronic acid, polyvinylpyrrolidone, polyvinyl alcohol, polylactic acid (PLA), polyglycolic acid (PGA), polylactive co-glycolic acid (PLGA), polyethylene glycol (PEG, PEG 300, PEG 400, PEG 600, PEG 3350, PEG 4000), cellulose, methylcellulose, carboxymethyl cellulose, sodium carboxymethyl cellulose, hydroxypropyl methylcellulose, acacia, lecithin, Polysorbate 20, Polysorbate 80, Pluronic® F-68, Sorbitantrioleate (Span® 85), EDTA, hydroxypropyl cellulose, sodium chloride, sodium phosphate, ammonium acetate, potassium phosphate, sodium citrate, sodium hydroxide, sodium carbonate, Tris base-65, Tris acetate, Tris HCl-65, citrate buffer, talc, silica, fats, methyl paraben, propyl paraben, selenium, vitamins (A, E, C, retinyl palmitate, and selenium), amino acids (methionine, cysteine, arginine), citric acid, sodium citrate, benzyl alcohol, chlorbutanol, cresol, phenol, thimerosal, EDTA, acetone sodium bisulfate, ascorbyl palmitate, ascorbate, castor oil, cottonseed oil, alum, aluminum hydroxide, aluminum phosphate, calcium phosphate hydroxide, paraffin oil, squalene, Quil IL-1, IL-2, IL-12, Freund's complete adjuvant, Freund's incomplete adjuvant, killed Bordetella pertussis, Mycobacterium bovis, and toxoids. The one or more selected excipients may be selected to improve the stability of the substance of interest during drying and storage of the microneedle devices, as well providing bulk and/or mechanical properties to the microneedle array and/or serve as an adjuvant to improve the immune response to a vaccine.

The arrays of microneedles may be made by any methods known in the art. For example, the arrays of microneedles may be made using a molding process, which advantageously is highly scalable. The process may include filling a mold with fluidized materials; drying the fluidized material to form the microneedles, the predefined fracture regions if included, and base substrate; and then removing the formed part from the mold. These filling and drying steps may be referred to as “casting” in the art. In an aspect, the methods for making the microneedles are performed under a minimum ISO 7 (class 10,000) process or an ISO 5 (class 100) process.

In one embodiment, the manufacture of solid, bioerodible microneedles includes filling a negative mold of the one or more microneedles with an aqueous or non-aqueous casting solution of the substance of interest and drying the casting solution to provide the one or more solid microneedles. In other embodiments, other solvent or solventless systems may be used. Non-limiting examples of methods for filling the negative mold include deposition, coating, printing, spraying, and microfilling techniques. The casting solution may be dried or cured at ambient temperature, under refrigeration, or at temperatures above ambient (e.g., 30 to 60° C., or higher) for a period from about 5 seconds to about one week to form the dry solid microneedles. In some embodiments, the dry or cure time is from about 10 seconds to about 24 hours, from about 30 minutes to about 12 hours, from about 10 minutes to about 1 hour, or from about 1 minute to about 30 minutes. In a preferred embodiment, the dry or cure time is from about 10 seconds to about 30 minutes.

Alternatively, the casting solution may be vacuum-filled or filled into the mold using a combination of non-vacuum filling and vacuum-filling. For example, in an embodiment the negative mold comprises a non-porous but gas-permeable material (e.g., PDMS) through which a backside vacuum can be applied. Although the negative mold is solid, it was determined that a sufficient vacuum could be applied through the backside when the molds are formed of such materials. In some embodiments, the backside vacuum may be used alone or in combination with a positive pressure applied on top of the mold for quicker filling. Such embodiments could advantageously reduce the time required and improve the accuracy and completeness when filling the mold with casting solution. For example, the casting solution may be vacuum-filled using a backside vacuum for a period from about 3 minutes to about 6 hours, from about 3 minutes to about 3 hours, from about 3 minutes to about 1 hour, or from about 3 minutes to about 30 minutes.

Although various temperatures and humidity levels can be employed to dry the casting solution, the formulations preferably are dried at temperature from about 1° C. to about 150° C. (e.g., from about 5° C. to about 99° C., from about 15° C. to about 45° C., from about 25° C. to about 45° C., or at about ambient temperature) and about 0 to about 40% relative humidity, e.g., about 0% to about 20% relative humidity.

In some embodiments, it may be desirable to use a multi-step casting process to form the microneedles and substrate. For example, the tips of the microneedles may be partially filled in a first step with a casting solution comprising the substance of interest followed by one or more subsequent fill steps with casting solutions of bulking polymers with or without the same or a different substance of interest. After filling and at least partially drying the microneedles in the negative mold, the adhesive layer and backing layer may be applied to the base substrate prior to removing the microneedles from the mold. In some embodiments, the adhesive layer and/or backing layer are pre-formed prior to application to the base substrate, while in other embodiments the adhesive layer and/or backing layer may be formed directly in-line.

In one embodiment, the multi-step casting process includes (1) a first cast of immunogenic particles in excipient forming the microneedles, (2) a second cast of a frangible material forming a fracture region, and (3) a third cast of a matrix material forming the backing and/or base substrate.

After at least partially drying the microneedles, the microneedles may be removed from the mold. For example, the microneedles may be removed from the mold before fully dry (e.g., when still in a rubbery state), but when strong enough to be peeled, and then dried further once removed from the mold to further solidify/harden the microneedles. Such a technique may be useful when carboxymethylcellulose sodium, polyvinyl alcohol, sugars, and other materials are used as a bulking polymer (matrix material) in the microneedles. In such embodiments, the microneedles may complete drying prior to or after packaging.

In embodiments, the drug delivery devices include a predefined fracture region. The substrate and/or one or more microneedles may include the predefined fracture region. In embodiments, this region may be considered to be a frangible interface between the microneedles and the substrate. The predefined fracture region may increase the likelihood that the microneedles or the microneedles and a portion of the substrate separate at or near a desired location. The predefined fracture region, in some embodiments, ensures that the microneedles or the microneedles and a portion of the substrate separate at or near a desired location.

In embodiments, the predefined fracture region comprises a structural or physical feature (i.e., a geometric feature) that increases the likelihood that the separation of the one or more microneedles will occur at a desired location, for example, where the force required to separate the microneedle from the substrate is greater in the perpendicular direction and less in the lateral direction. For example, the predefined fraction region may include a substantially narrowed portion, a scored portion, a notched portion, an interface of different materials, or a combination thereof. An interface of different materials may be provided by forming at least a portion of the substrate and at least a portion of the one or more microneedles from different materials or combinations of materials.

In one embodiment, the microneedle patch device includes a system for triggering, after the microneedles are inserted at least partially into a biological tissue, a change in rate of release of the immunogenic particles from the microneedles and into the biological tissue.

In embodiments, the system for triggering change in the rate of release of immunogenic particle is a barrier that may be positioned in or on at least part of the microneedle to impede release of the drug from the microneedle in at least one direction and/or for a predetermined period of time. In one embodiment, the barrier is configured to permit (i) discrete periods of drug release upon or after implantation, (ii) control of the region of the microneedles from which the drug is released, or (iii) a combination thereof. The term “barrier” and the phrase “barrier material” are used interchangeably herein.

In embodiments, the barrier impedes release of the immunogenic particles from the microneedle until the barrier no longer obstructs release of the immunogenic particles. The obstruction provided by the barrier may be permanent or lessened gradually or substantially instantaneously.

A barrier generally may be positioned in a microneedle, on a microneedle, or a combination thereof. For example, a barrier may at least partially encapsulate immunogenic particles in a microneedle, be dispersed within the matrix of one or more microneedles, be positioned on and/or at the surface of one or more microneedles, or a combination thereof. When dispersed within the matrix of one or more microneedles, the barrier may include discrete regions within the matrix.

The microneedle patch device optionally includes a supporting layer adhered to the substrate. The supporting layer may be adhered to the substrate by any means known in the art, including an adhesive. In one embodiment, an adhesive layer is used to adhere the supporting layer to the substrate.

The supporting layer may be made out of a variety of materials. In some embodiments, the supporting layer may be a composite material or multilayer material including materials with various properties to provide the desired properties and functions. For example, the supporting layer may be flexible, semi-rigid, or rigid, depending on the particular application. As another example, the supporting layer may be substantially impermeable, protecting the one or more microneedles (or other components) from moisture, gases, and contaminants.

Alternatively, the supporting layer may have other degrees of permeability and/or porosity based on the desired level of protection that is desired. Non-limiting examples of materials that may be used for the supporting layer include various polymers, elastomers, foams, paper-based materials, foil-based materials, metallized films, and non-woven and woven materials.

An optional mechanical force indicator may be disposed between the supporting layer and the substrate, or it may be located within or be an integral part of the supporting layer. The mechanical force indicator may be used to indicate to a person the amount of force and/or pressure applied to the drug delivery device during its use. For example, in one embodiment, the indicator is configured to provide a signal when a force applied to the drug delivery device by a person (in the course of applying the drug delivery device to a patient's skin to insert the one or more microneedles into the patient's skin) meets or exceeds a predetermined threshold. The predetermined threshold may be the minimum force or some amount greater than the minimum force that is required for a particular drug delivery device to be effectively applied to a patient's skin. In other words, it may be the force needed to cause the microneedles to be properly, e.g., partially or fully, inserted into a patient's skin; or it may be the force needed to cause the microneedles to be properly, e.g., partially or fully, inserted into a patient's skin, and separate the microneedles from the substrate.

In embodiments, the microneedle patch devices provided herein include a housing. At least one of the substrate and supporting layer may be associated with the housing in any manner. For example, at least one of the substrate or supporting layer may be disposed in the housing. As a further example, at least one of the substrate and supporting layer may be fixably or movably mounted in or on the housing by any means known in the art. For example, the substrate and/or supporting layer, when movably mounted, may be mounted on tracks, a central axis, or a combination thereof.

The housing may include a portion configured to accommodate the application of a force. In one embodiment, the portion configured to accommodate the application of a force is a depressible portion. The depressible portion generally may be any portion of the housing configured to transfer a force applied to the device to the substrate. For example, the depressible portion may include a piston-like apparatus movably mounted in the housing. In another example, the depressible portion may include an elastic portion of the housing that is depressible upon application of a force. The depressible portion may or may not contact the supporting layer and/or substrate prior to application of a force.

The depressible portion, in embodiments, imparts a shearing force to the substrate upon application of an input force. In some embodiments, the input force could be applied directly to the supporting layer which in turn imparts an output force to the substrate.

The depressible portion, in embodiments, applies a shearing force to the supporting layer and/or substrate by directly contacting the supporting layer and/or substrate. In one embodiment, at least a portion of the depressible portion that contacts the supporting layer and/or substrate is configured to impart motion to the supporting layer and/or substrate upon contact. In another embodiment, at least a portion of the depressible portion that contacts the supporting layer and/or substrate, and at least a portion of the supporting layer and/or substrate that contacts the depressible portion is configured to impart motion to the supporting layer and/or substrate. The contacting portions of the depressible portion, substrate, supporting layer, or a combination thereof may be angled, non-linear, etc., and the contacting surfaces may be lubricated and/or coated or constructed with a material that promotes the motion of the supporting layer and/or substrate.

The immunogenic particles in the composition comprise a multilayer film, the multilayer film comprising two or more layers of charged polyelectrolytes, wherein adjacent layers comprise oppositely charged polyelectrolytes, one of the charged polyelectrolyte layers in the multilayer film comprises an antigenic polyelectrolyte comprising a peptide epitope covalently linked to the antigenic polyelectrolyte, wherein the polyelectrolytes that are not the antigenic polyelectrolyte comprise a polycationic material or a polyanionic material having a molecular weight of greater than 1,000 and at least 5 charges per molecule, and wherein the multilayer film is deposited on a core particle or forms a hollow particle to provide the composition.

More specifically, polyelectrolyte multilayer films are thin films (e.g., a few nanometers to micrometers thick) composed of alternating layers of oppositely charged polyelectrolytes. Such films can be formed by layer-by-layer assembly on a substrate. In electrostatic layer-by-layer self-assembly (“ELBL”), the physical basis of association of polyelectrolytes is electrostatic attraction. Film buildup is possible because the sign of the surface charge density of the film reverses on deposition of successive layers. The generality and relative simplicity of the ELBL film process permits the deposition of many different types of polyelectrolyte onto many different types of surface. Polypeptide multilayer films are a subset of polyelectrolyte multilayer films, comprising at least one layer comprising a charged polypeptide, herein referred to as a designed polypeptide. A key advantage of polypeptide multilayer films over films made from other polymers is their biocompatibility. ELBL films can also be used for encapsulation. Applications of polypeptide films and microcapsules include, for example, nano-reactors, biosensors, artificial cells, and drug delivery vehicles.

The term “polyelectrolyte” includes polycationic and polyanionic materials having a molecular weight of greater than 1,000 and at least 5 charges per molecule. Suitable polycationic materials include, for example, polypeptides and polyamines. Polyamines include, for example, a polypeptide such as poly-L-lysine (PLL) or poly-L-ornithine, polyvinyl amine, poly(aminostyrene), poly(aminoacrylate), poly (N-methyl aminoacrylate), poly (N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), poly (diallyl dimethylammonium chloride), poly(N,N,N-trimethylaminoacrylate chloride), poly(methyacrylamidopropyltrimethyl ammonium chloride), chitosan and combinations comprising one or more of the foregoing polycationic materials. Suitable polyanionic materials include, for example, a polypeptide such as poly-L-glutamic acid (PGA) and poly-L-aspartic acid, a nucleic acid such as DNA and RNA, alginate, carrageenan, furcellaran, pectin, xanthan, hyaluronic acid, heparin, heparan sulfate, chondroitin sulfate, dermatan sulfate, dextran sulfate, poly(meth)acrylic acid, oxidized cellulose, carboxymethyl cellulose, acidic polysaccharides, and croscarmelose, synthetic polymers and copolymers containing pendant carboxyl groups, and combinations comprising one or more of the foregoing polyanionic materials. In one embodiment, the RSV epitope and the polyelectrolyte have the same sign of charge.

In an aspect, a stable multilayer film is a film that once formed, retains more than half its components after incubation at in PBS at 37° C. for 24 hours.

In an aspect, the antigenic polyelectrolyte is in the outermost later of the multilayer film.

An antigenic polyelectrolyte is a polyelectrolyte comprising a peptide antigen. In an aspect, the antigenic polyelectrolyte is a polypeptide such as a designed polypeptide.

A designed polypeptide means a polypeptide including a peptide antigen that has sufficient charge for stable binding to an oppositely charged surface, that is, a polypeptide that can be deposited into a layer of a multilayer film wherein the driving force for film formation is electrostatics. In another embodiment, the solubility of the designed polypeptide at pH 4 to 10 is greater than or equal to about 0.1 mg/mL. In another embodiment, the solubility of the designed polypeptide at pH 4 to 10 is greater than or equal to about 1 mg/mL. The solubility is a practical limitation to facilitate deposition of the polypeptides from aqueous solution. A practical upper limit on the degree of polymerization of an antigenic polypeptide is about 1,000 residues. It is conceivable, however, that longer composite polypeptides could be realized by an appropriate method of synthesis.

In specific embodiments, the magnitude of the net charge per residue of the designed polypeptide is greater than or equal to 0.1, 0.2, 0.3, 0.4 or 0.5 at pH 7.0. In one embodiment, the ratio of the number of charged residues of the same polarity minus the number of residues of the opposite polarity to the total number of residues in the polypeptide is greater than or equal to 0.5 at pH 7.0. In other words, the magnitude of the net charge per residue of the polypeptide is greater than or equal to 0.5. While there is no absolute upper limit on the length of the polypeptide, in general, designed polypeptides suitable for ELBL deposition have a practical upper length limit of 1,000 residues. Designed polypeptides can include sequences found in nature such as RSV epitopes as well as regions that provide functionality to the peptides such as charged regions also referred to herein as surface adsorption regions, which allow the designed polypeptides to be deposited into a polypeptide multilayer film.

Positively-charged (basic) naturally-occurring amino acids at pH 7.0 are arginine (Arg), histidine (His), omithine (Om), and lysine (Lys). Negatively-charged (acidic) naturally-occurring amino acid residues at pH 7.0 are glutamic acid (Glu) and aspartic acid (Asp). A mixture of amino acid residues of opposite charge can be employed so long as the overall net ratio of charge meets the specified criteria. In one embodiment, a designed polypeptide is not a homopolymer. In another embodiment, a designed polypeptide is unbranched.

In one embodiment, the multilayer film is deposited on a core particle, such as a CaCO3 nanoparticle, a latex particle, or an iron particle. Particle sizes on the order of 5 nanometers (nm) to 50 micrometers (um) in diameter are particularly useful. Particles made of other materials can also be used as cores provided that they are biocompatible, have controllable size distribution, and have sufficient surface charge (either positive or negative) to bind polyelectrolyte peptides. Examples include nanoparticles and microparticles made of materials such as polylactic acid (PLA), polylactic acid glycolic acid copolymer (PLGA), polyethylene glycol (PEG), chitosan, hyaluronic acid, gelatin, or combinations thereof. Core particles could also be made of materials that are believed to be inappropriate for human use provided that they can be dissolved and separated from the multilayer film following film fabrication. Examples of the template core substances include organic polymers such as latex or inorganic materials such as silica.

One design concern is control of the stability of polypeptide ELBL films. Ionic bonds, hydrogen bonds, van der Waals interactions, and hydrophobic interactions contribute to the stability of multilayer films. In addition, covalent disulfide bonds formed between sulfhydryl-containing amino acids in the polypeptides within the same layer or in adjacent layers can increase structural strength. Sulfydryl-containing amino acids include cysteine and homocysteine and these residues can be readily incorporated into synthetic designed peptides. In addition, sulfhydryl groups can be incorporated into polyelectrolyte homopolymers such as poly-L-lysine or poly-L-glutamic acid by methods well described in the literature. Sulfhydryl-containing amino acids can be used to “lock” (bond together) and “unlock” layers of a multilayer polypeptide film by a change in oxidation potential. Also, the incorporation of a sulfhydryl-containing amino acid in a designed polypeptide enables the use of relatively short peptides in thin film fabrication, by virtue of intermolecular disulfide bond formation.

In one embodiment, the designed sulfhydryl-containing polypeptides, whether synthesized chemically or produced in a host organism, are assembled by ELBL in the presence of a reducing agent to prevent premature disulfide bond formation. Following film assembly, the reducing agent is removed, and an oxidizing agent is added. In the presence of the oxidizing agent disulfide bonds form between sulfhydryls groups, thereby “locking” together the polypeptides within layers and between layers where thiol groups are present. Suitable reducing agents include dithiothreitol (DTT), 2-mercaptoethanol (BME), reduced glutathione, tris(2-carboxyethyl)phosphine hydrochloride (TCEP), and combinations of more than one of these chemicals. Suitable oxidizing agents include oxidized glutathione, tert-butylhydroperoxide (t-BHP), thimerosal, diamide, 5,5′-dithio-bis-(2-nitro-benzoic acid) (DTNB), 4,4′-dithiodipyridine, sodium bromate, hydrogen peroxide, sodium tetrathionate, porphyrindin, sodium orthoiodosobenzoate, and combinations of more than one of these chemicals.

As an alternative to disulfide bonds, chemistries that produce other covalent bonds can be used to stabilize ELBL films. For films comprised of polypeptides, chemistries that produce amide bonds are particularly useful. In the presence of appropriate coupling reagents, acidic amino acids (those with side chains containing carboxylic acid groups such as aspartic acid and glutamic acid) will react with amino acids whose side chains contain amine groups (such as lysine and ornithine) to form amide bonds. Amide bonds are more stable than disulfide bonds under biological conditions and amide bonds will not undergo exchange reactions. Many reagents can be used to activate polypeptide side chains for amide bonding. Carbodiimide reagents, such as the water soluble 1-ethyl-3-(3-dimethylaminopropyl) carbodiimide (EDC) will react with aspartic acid or glutamic acid at slightly acidic pH, forming an intermediate product that will react irreversibly with an amine to produce an amide bond. Additives such as N-hydroxysuccinimide are often added to the reaction to accelerate the rate and efficiency of amide formation. After the reaction the soluble reagents are removed from the nanoparticles or microparticles by centrifugation and aspiration. Examples of other coupling reagents include diisopropylcarbodiimide, HBTU, HATU, HCTU, TBTU, and PyBOP. Examples of other additives include sulfo-N-hydroxysuccinimide, 1-hydroxbenzotriazole, and 1-hydroxy-7-aza-benzotriazole. The extent of amide cross linking can be controlled by modulating the stoichiometry of the coupling reagents, the time of reaction, or the temperature of the reaction, and can be monitored by techniques such as Fourier transform—infrared spectroscopy (FT-IR).

Covalently cross-linked ELBL films have desirable properties such as increased stability. Greater stability allows for more stringent conditions to be used during nanoparticle, microparticle, nanocapsule, or microcapsule fabrication. Examples of stringent conditions include high temperatures, low temperatures, cryogenic temperatures, high centrifugation speeds, high salt buffers, high pH buffers, low pH buffers, filtration, and long term storage.

As used herein, a peptide epitope includes the epitopes described in U.S. Pat. No. 7,615,530, incorporated herein by reference in its entirety.

In one embodiment, the peptide epitope comprises a viral antigen. Suitable viral antigens include, but are not limited to, retroviral antigens such as HIV-1 antigens including the gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the attachment (G), fusion (F) and matrix (M2) proteins and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, 80% E, and other Japanese encephalitis viral antigen components; rabies viral antigens such as rabies glycoprotein, rabies nucleoprotein and other rabies viral antigen components; and combinations comprising one or more of the foregoing antigenic determinant regions.

In another embodiment, the peptide epitope comprises a bacterial antigen. Suitable bacterial antigens include, but are not limited to, pertussis bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens; Mycobacterium tuberculosis bacterial antigens such as heat shock protein 65 (HSP65), the 30 kDa major secreted protein, antigen 85A and other mycobacterial antigen components; Helicobacter pylori bacterial antigen components; pneumococcal bacterial antigens such as pneumolysin, and other pneumococcal bacterial antigen components; haemophilus influenza bacterial antigens; anthrax bacterial antigens such as anthrax protective antigen and other anthrax bacterial antigen components; rickettsiae bacterial antigens such as romps and other rickettsiae bacterial antigen components; and combinations comprising one or more of the foregoing antigenic determinant regions.

In another embodiment, the peptide epitope comprises a fungal antigen. Suitable fungal antigens include, but are not limited to, candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components, and tinea fungal antigens such as trichophytin and other coccidiodes fungal antigen components; and combinations comprising one or more of the foregoing antigenic determinant regions.

In another embodiment, the peptide epitope region comprises a parasite antigen. Suitable protozoal and other parasitic antigens include, but are not limited to, Plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen and other trypanosomal antigen components; and combinations comprising one or more of the foregoing parasite antigens.

In one embodiment, the peptide epitope is from respiratory syncytial virus, such as an epitope from the attachment (G) protein and its subunits, the fusion (F) protein and its subunits, and the matrix (M2) protein and its subunits. In another embodiment, the polypeptide epitope is from influenza virus such as an epitope from the hemaglutinin (HA) protein and its subunits, the neuraminidase (NA) protein and its subunits, or the matrix protein ectodomain (M2). In another embodiment, the polypeptide epitope is from the malaria parasite, such Plasmodium falciparum, P. vivax, P. ovale and P. malariae, and including, for example the circumsporozoite (CS) protein and subunits including T1, B and T* epitopes.

As used herein, the Plasmodium falciparum circumsporozoite protein antigens are as described in U.S. Pat. No. 9,433,671:

(SEQ ID NO: 1)
T1: DPNANPNVDPNANPNV
(SEQ ID NO: 2)
B: NANP
(SEQ ID NO: 3)
T*: EYLNKIQNSLSTEWSPCSVT

In certain embodiments, the T, B or T* epitope, particularly the B epitope, is repeated 2 or more times.

The T* epitope can be a modified T* epitope as described in U.S. Pat. No. 9,968,665:

(SEQ ID NO: 4)
EYLNKIQNSLSTEWSPSSVT,
or
(SEQ ID NO: 5)
EYLNKIQNSLSTEWSPASVT.

RSV epitopes reside in sequences of the RSV-G, RSV-F or RSV-M2 proteins as described in U.S. Pat. No. 9,487,593. The amino acid sequences of the full-length proteins are as follows: (selected peptide epitopes are underlined)

| | RSV-G- | |
|---|---|---|
| SEQ ID NO: 6 | | |
| 1 | MSKNKDQRTA KTLERTWDTL NHLLFISSCL YKLNLKSVAQ ITLSILAMII STSLIIAAII | 60 |
| 61 | FIASANHKVT PTTAIIQDAT SQIKNTTPTY LTQNPQLGIS PSNPSEITSQ ITTILASTTP | 120 |
| 121 | GVKSTLQSTT VKTKNTTTTQ TQPSKPTTKQ RQNKPPSKPN NDFHFEVFNF VPCSICSNNP | 180 |
| 181 | TCWAICKRIP NKKPGKKTTT KPTKKPTLKT TKKDPKPQTT KSKEVPTTKP TEEPTINTTK | 240 |
| 241 | TNIITTLLTS NTTGNPELTS QMETFHSTSS EGNPSPSQVS TTSEYPSQPS SPPNTPRQ | 298 |
| | RSV-F- | |
| SEQ ID NO: 7 | | |
| 1 | MELLILKANA ITTILTAVTF CFASGQNITE EFYQSTCSAV SKGYLSALRT GWYTSVITIE | 60 |
| 61 | LSNIKENKCN GTDAKVKLIK QELDKYKNAV TELQLLMQST PPTNNRARRE LPRFMNYTLN | 120 |
| 121 | NAKKTNVTLS KKRKRRFLGF LLGVGSAIAS GVAVSKVLHL EGEVNKIKSA LLSTNKAVVS | 180 |
| 181 | LSNGVSVLTS KVLDLKNYID KQLLPIVNKQ SCSISNIETV IEFQQKNNRL LEITREFSVN | 240 |
| 241 | AGVTTPVSTY MLTNSELLSL INDMPITNDQ KKLMSNNVQI VRQQSYSIMS IIKEEVLAYV | 300 |
| 301 | VQLPLYGVID TPCWKLHTSP LCTTNTKEGS NICLTRTDRG WYCDNAGSVS EFPQAETCKV | 360 |
| 361 | QSNRVFCDTM NSLTLPSEIN LCNVDIFNPK YDCKIMTSKT DVSSSVITSL GAIVSCYGKT | 420 |
| 421 | KCTASNKNRG IIKTFSNGCD YVSNKGMDTV SVGNTLYYVN KQEGKSLYVK GEPIINFYDP | 480 |
| 481 | LVFPSDEFDA SISQVNEKIN QSLAFIRKSD ELLHNVNAGK STTNIMITTI IIVIIVILLS | 540 |
| 541 | LIAVGLLLYC KARSTPVTLS KDQLSGINNI AFSN | 574 |
| | RSV-M- | |
| SEQ ID NO: 8 | | |
| 1 | MSRRNPCKFE IRGHCLNGKR CHFSHNYFEW PPHALLVRQN FMLNRILKSM DKSIDTLSEI | 60 |
| 61 | SGAAELDRTE EYALGVVGVL ESYIGSINNI TKQSACVAMS KLLTELNSDD IKKLRDNEEL | 120 |
| 121 | NSPKIRVYNT VISYIESNRK NNKQTIHLLK RLPADVLKKT IKNTLDIHKS ITINNPKEST | 180 |
| 181 | DTNDHAKN NDTT | 194 |

In an aspect, the multilayer films also include a toll-like receptor ligand, or TLR ligand. The TLR ligand can be covalently linked to the antigenic polyelectrolyte. TLR ligands are molecules that bind to TLRs and either activate or repress TLR receptors. Activation of TLR signaling through recognition of pathogen-associated molecular patterns (PAMPs) and mimics leads to the transcriptional activation of genes encoding pro-inflammatory cytokines, chemokines and co-stimulatory molecules, which can control the activation of the antigen-specific adaptive immune response. TLRs have been pursued as potential therapeutic targets for various inflammatory diseases and cancer. Following activation, TLRs induce the expression of a number of protein families, including inflammatory cytokines, type I interferons, and chemokines. TLR receptor ligands can function as adjuvants for the immune response.

Exemplary TLR ligands include a TLR1 ligand, a TLR2 ligand, a TLR3 ligand, a TLR4 ligand, a TLR5 ligand, a TLR6 ligand, a TLR 7 ligand, a TLR8 ligand, a TLR9 ligand and combinations thereof.

Exemplary TLR1 ligands include triacyl bacterial lipoproteins such as Pam3Cys ([N-palmitoyl-S[2,3-bis(palmitoyloxy)propyl]cysteine]). Exemplary TLR2 ligands include diacyl bacterial lipoproteins such as Pam2Cys (Pam2Cys [S-[2,3-bis(palmitoyloxy)propyl]cysteine1), mycoplasmal macrophage-activating lipopeptide-2 (MALP2), or zymosan (fungal). Exemplary TLR6 ligands are diacyl lipopeptides. TLR1 and TLR6 require heterodimerization with TLR2 to recognize ligands. TLR1/2 are activated by triacyl lipoprotein (or a lipopeptide, such as Pam3Cys), whereas TLR6/2 are activated by diacyl lipoproteins (e.g., Pam2Cys), although there may be some cross-recognition.

An exemplary TLR3 ligand is Poly(I:C). Exemplary TLR4 ligands are lipopolysaccharide (LPS), monophospholipid A (MPLA), fusion protein of respiratory syncytial virus, and envelope protein of mouse mammary tumor virus. An exemplary TLR5 ligand is flagellin. Exemplary TLR7 ligands are nucleoside analogs such as loxoribine (guanosine analog) and imidazoquinolines such as imiquimod and R848. An exemplary TLR8 ligand is single-stranded RNA. An exemplary TLR9 ligand is unmethylated CpG Oligodeoxynucleotide DNA.

In one embodiment, an antigenic polyelectrolyte, e.g., an antigenic polypeptide, has a TLR ligand covalently attached thereto. For example, Pam3Cys can be covalently coupled to a polypeptide chain by standard polypeptide synthesis chemistry. In one embodiment, Pam3Cys is covalently linked to an antigenic polypeptide through direct covalent linkage via an amide bond formed between the carboxylic acid of Pam3Cys-OH (commercially available from Bachem, Inc.) to the N-terminal of a peptide. A convenient way to accomplish this reaction is to couple Pam3Cys-OH in the presence of an amide bond forming reagent such as HBTU (0-Benzotriazole-N,N,N′,N′-tetramethyl-uronium-hexafluoro-phosphate), HATU (2-(1H-7-Azabenzotriazol-1-yl)-1,1,3,3-tetramethyluronium hexafluorophosphate Methanaminium), or DIPCDI (N,N′-Diisopropylcarbodiimide) to a synthetic peptide on a solid phase synthesis resin bead. The progress of the coupling reaction can be monitored colorimetrically by ninhydrin assay and, following completion, excess Pam3Cys-OH and other reagents can be washed away. The synthetic Pam3Cys peptide conjugate is cleaved from the resin and purified by chromatography. For example, Pam3Cys peptides can be purified by reverse phase HPLC using a C4 column and a water/isopropanol gradient. An advantage of this approach is that the Pam3Cys/antigenic polypeptide is strictly controlled in a 1:1 ratio.

In another embodiment, Pam3Cys-OH is conjugated specifically to the side chain c-amine of lysine residue, either specifically to a resin bound peptide as described above, or nonspecifically to an unprotected peptide or protein using water soluble coupling reagent such as EDC/sulfo-NHS. The product of that reaction is purified, for example, by gel permeation chromatography or dialysis, then incorporated into a particle by LBL or other methods.

In yet another embodiment, Pam3Cys-OH is conjugated to a highly charged polyelectrolyte such as polylysine and then incorporated into an LBL film along with one or more designed peptides. Thus, Pam3Cys, for example, is amide conjugated to a sequence containing a surplus of charge such as a polylysine segment of about four to about forty residues in length and purified as described above, or in the case of Pam3Cys-Ser-Lys-Lys-Lys-Lys-OH (Pam3Cys-SK4) purchased from a commercial vendor (EMD Biosciences). Peptides such as these could be incorporated into a film in a step before, during, or after incorporation of the antigenic determinant region. The advantage of this approach would be that only one or (or perhaps several) Pam3Cys polyelectrolyte peptides could be used in any combination with antigenic designed polypeptides, greatly simplifying synthesis. In addition, the Pam3Cys/antigenic designed polypeptide stoichiometry can be varied as desired to optimize potency or minimize toxicities.

In yet another embodiment, commercially available Pam3Cys reagents Pam3Cys-OH or Pam3Cys-SK4 could be incorporated into particles directly through a non-LBL process. These include during particle precipitation (for example during the precipitation of core particles such as CaCO3), particle fabrication (for example during water-in-oil dispersion of PLGA), or liposome fabrication. Finally, it is possible that the hydrophobicity of the Pam3Cys could drive adsorption to a surface. Thus, simple incubation of particles in Pam3Cys-OH or Pam3Cys-SK4 solutions could result in an antigenic particle with incorporated TLR-2 ligand.

In another embodiment, conjugation of monophosphoryl lipid A (MPLA) to a designed peptide is possible and appropriate chemistries are known in the art. These chemistries allow for the specific conjugation of MPLA derivatives to modified DPs via the azide/alkyne cycloaddition reaction (click chemistry), which occurs readily and efficiently in aqueous buffers (Guo et al. US20090239378, incorporated herein by reference). Tumor associated carbohydrate antigen conjugates to MPLA have been made using this technology and resulting conjugates shown to be immunogenic in mice.

Alternatively, due to its highly hydrophobic nature MPLA will adsorb efficiently to surfaces. Thus, a dilute solution of MPLA, for example 10-100 μg/mL in dilute neutral aqueous buffers will adsorb to a suspension of CaCO3 microparticles coated with designed peptide films. The efficiency of the loading process can be monitored either by chemical methods or by a cell-based bioassay.

A method of making a polyelectrolyte multilayer film comprises depositing a plurality of layers of oppositely charged chemical species on a substrate. At least one layer, preferably the outermost layer, comprises an antigenic polyelectrolyte as described herein. Successively deposited polyelectrolytes will have opposite net charges. In one embodiment, deposition of a polyelectrolyte comprises exposing the substrate to an aqueous solution comprising a polyelectrolyte at a pH at which it has a suitable net charge for ELBL. In other embodiments, the deposition of a polyelectrolyte on the substrate is achieved by sequential spraying of solutions of oppositely charged polypeptides. In yet other embodiments, deposition on the substrate is by simultaneous spraying of solutions of oppositely charged polyelectrolytes.

In the ELBL method of forming a multilayer film, the opposing charges of the adjacent layers provide the driving force for assembly. It is not critical that polyelectrolytes in opposing layers have the same net linear charge density, only that opposing layers have opposite charges. One standard film assembly procedure by deposition includes forming aqueous solutions of the polyions at a pH at which they are ionized (i.e., pH 4-10), providing a substrate bearing a surface charge, and alternating immersion of the substrate into the charged polyelectrolyte solutions. The substrate is optionally washed in between deposition of alternating layer.

The concentration of polyelectrolyte suitable for deposition of the polyelectrolyte can readily be determined by one of ordinary skill in the art. An exemplary concentration is 0.1 to 10 mg/mL. For typical non-polypeptide polyelectrolytes such as poly(acrylic acid) and poly(allylamine hydrochloride), typical layer thicknesses are about 3 to about 5 Å, depending on the ionic strength of solution. Short polyelectrolytes typically form thinner layers than long polyelectrolytes. Regarding film thickness, polyelectrolyte film thickness depends on humidity as well as the number of layers and composition of the film. For example, PLL/PGA films 50 nm thick shrink to 1.6 nm upon drying with nitrogen. In general, films of 1 nm to 100 nm or more in thickness can be formed depending on the hydration state of the film and the molecular weight of the polyelectrolytes employed in the assembly.

In addition, the number of layers required to form a stable polyelectrolyte multilayer film will depend on the polyelectrolytes in the film. For films comprising only low molecular weight polypeptide layers, a film will typically have 4 or more bilayers of oppositely charged polypeptides. For films comprising high molecular weight polyelectrolytes such as poly(acrylic acid) and poly(allylamine hydrochloride), films comprising a single bilayer of oppositely charged polyelectrolyte can be stable. Studies have shown that polyelectrolyte films are dynamic. The polyelectrolytes contained within a film can migrate between layers and can exchange with soluble polyelectrolytes of like charge when suspended in a polyelectrolyte solution. Moreover polyelectrolyte films can disassemble or dissolve in response to a change in environment such as temperature, pH, ionic strength, or oxidation potential of the suspension buffer. Thus, some polyelectrolytes and particularly peptide polyelectrolytes exhibit transient stability. The stability of peptide polyelectrolyte films can be monitored by suspending the films in a suitable buffer under controlled conditions for a fixed period of time, and then measuring the amounts of the peptides within the film with a suitable assay such as amino acid analysis, HPLC assay, or fluorescence assay. Peptide polyelectrolyte films are most stable under conditions that are relevant to their storage and usage as vaccines, for example in neutral buffers and at ambient temperatures such as 4° C. to 37° C. Under these conditions stable peptide polyelectrolyte films will retain most of their component peptides for at least 24 hours and often up to 14 days and beyond.

For synthesis of antigenic polypeptides, each of the independent regions of the antigenic polypeptide can be synthesized separately by solution phase peptide synthesis, solid phase peptide synthesis, or genetic engineering of a suitable host organism. Solution phase peptide synthesis is the method used for production of most of the approved peptide pharmaceuticals on the market today. A combination of solution phase and solid phase methods can be used to synthesize relatively long peptides and even small proteins. Peptide synthesis companies have the expertise and experience to synthesize difficult peptides on a fee-for-service basis. The syntheses are performed under good manufacturing practices (GMP) conditions and at a scale suitable for clinical trials and commercial drug launch.

Alternatively, the various independent regions can be synthesized together as a single polypeptide chain by solution-phase peptide synthesis, solid phase peptide synthesis or genetic engineering of a suitable host organism. The choice of approach in any particular case will be a matter of convenience or economics.

If the various epitopes and surface adsorption regions (e.g., charged regions) are synthesized separately, once purified, for example, by ion exchange chromatography or by high performance liquid chromatography, they are joined by peptide bond synthesis. That is, the N-terminus of the surface adsorption region and the C-terminus of the epitope are covalently joined to produce the designed polypeptide. Alternatively, the C-terminus of the surface adsorption region and the N-terminus of the epitope are covalently joined to produce the designed polypeptide. The individual fragments can be synthesized by solid phase methods and obtained as fully protected, fully unprotected, or partially protected segments. The segments can be covalently joined in a solution phase reaction or solid phase reaction. If one polypeptide fragment contains a cysteine as its N-terminal residue and the other polypeptide fragment contains a thioester or a thioester precursor at its C-terminal residue the two fragments will couple spontaneously in solution by a specific reaction commonly known (to those skilled in the art) as Native Ligation. Native Ligation is a particularly attractive option for designed peptide synthesis because it can be performed with fully deprotected or partially protected peptide fragments in aqueous solution and at dilute concentrations.

In one embodiment, the epitopes and/or surface adsorption regions are joined by peptidic or non-peptidic linkages as described in U.S. Pat. No. 7,723,294, incorporated herein by reference for its teaching of the use of non-peptidic linkages to join segments of polypeptides for use in multilayer films. Suitable non-peptidic linkers include, for example, alkyl linkers such as —NH—(CH2)s-C(O)—, wherein s=2-20. Alkyl linkers are optionally substituted by a non-sterically hindering group such as lower alkyl (e.g., C1-C6), lower acyl, halogen (e.g., Cl, Br), CN, NH2, phenyl, and the like. Another exemplary non-peptidic linker is a polyethylene glycol linker such as —NH—(CH2-CH2-O)n, —C(O)— wherein n is such that the linker has a molecular weight of 100 to 5000 Da, specifically 100 to 500 Da. Many of the linkers described herein are available from commercial vendors in a form suitable for use in solid phase peptide synthesis.

Exemplary microneedle patches include 0.1 ng to 10 micrograms of antigenic polyelectrolyte per patch.

The immunogenicity of an immunogenic composition may be enhanced in a number of ways. In one embodiment, the multilayer film optionally comprises one or more additional immunogenic bioactive molecules. Although not necessary, the one or more additional immunogenic bioactive molecules will typically comprise one or more additional antigenic determinants. Suitable additional immunogenic bioactive molecules include, for example, a drug, a protein, an oligonucleotide, a nucleic acid, a lipid, a phospholipid, a carbohydrate, a polysaccharide, a lipopolysaccharide, a low molecular weight immune stimulatory molecule, or a combination comprising one or more of the foregoing bioactive molecules. Other types of additional immune enhancers include a functional membrane fragment, a membrane structure, a virus, a pathogen, a cell, an aggregate of cells, an organelle, or a combination comprising one or more of the foregoing bioactive structures.

In one embodiment, the multilayer film optionally comprises one or more additional bioactive molecules. The one or more additional bioactive molecule can be a drug. Alternatively, the immunogenic composition is in the form of a hollow shell or a coating surrounding a core. The core comprises a variety of different encapsulants, for example, one or more additional bioactive molecules, including, for example, a drug. Thus, the immunogenic compositions designed as described herein could also be used for combined therapy, e.g., eliciting an immune response and for targeted drug delivery. Micron-sized “cores” of a suitable therapeutic material in “crystalline” form can be encapsulated by immunogenic composition comprising the antigenic polypeptides, and the resulting microcapsules could be used for drug delivery. The core may be insoluble under some conditions, for instance high pH or low temperature, and soluble under the conditions where controlled release will occur. The surface charge on the crystals can be determined by potential measurements (used to determine the charge in electrostatic units on colloidal particles in a liquid medium). The rate at which microcapsule contents are released from the interior of the microcapsule to the surrounding environment will depend on a number of factors, including the thickness of the encapsulating shell, the antigenic polypeptides used in the shell, the presence of disulfide bonds, the extent of cross-linking of peptides, temperature, ionic strength, and the method used to assemble the peptides. Generally, the thicker the capsule, the longer the release time.

In another embodiment, the additional immunogenic biomolecule is a nucleic acid sequence capable of directing host organism synthesis of a desired immunogen or interfering with the expression of genetic information from a pathogen. In the former case, such a nucleic acid sequence is, for example, inserted into a suitable expression vector by methods known to those skilled in the art. Expression vectors suitable for producing high efficiency gene transfer in vivo include retroviral, adenoviral and vaccinia viral vectors. Operational elements of such expression vectors include at least one promoter, at least one operator, at least one leader sequence, at least one terminator codon, and any other DNA sequences necessary or preferred for appropriate transcription and subsequent translation of the vector nucleic acid. In particular, it is contemplated that such vectors will contain at least one origin of replication recognized by the host organism along with at least one selectable marker and at least one promoter sequence capable of initiating transcription of the nucleic acid sequence. In the latter case, multiple copies of such a nucleic acid sequence will be prepared for delivery, for example, by encapsulation of the nucleic acids within a polypeptide multilayer film in the form of a capsule for intravenous delivery.

In construction of a recombinant expression vector, it should additionally be noted that multiple copies of the nucleic acid sequence of interest and its attendant operational elements may be inserted into each vector. In such an embodiment, the host organism would produce greater amounts per vector of the desired protein. The number of multiple copies of the nucleic acid sequence which may be inserted into the vector is limited only by the ability of the resultant vector due to its size, to be transferred into and replicated and transcribed in an appropriate host microorganism.

In one embodiment, the multilayer film/immunogenic composition evokes a response from the immune system to a pathogen. In one embodiment, a vaccine composition comprises an immunogenic composition in combination with a pharmaceutically acceptable carrier. Thus a method of vaccination against a pathogenic disease comprises the administering to a subject in need of vaccination an effective amount of the immunogenic composition.

As used herein, the phrase “penetrate a tissue surface” or the terms “penetrate” or “penetration” refers to the insertion of at least 50%, and typically substantially all, of the microneedles of an array of microneedles, including at least the tip or distal end portion of the microneedles, into a biological tissue. In a preferred embodiment, the “penetration” includes piercing the stratum corneum of the skin of a human patient such that at least the tip end portion of the microneedle is within or has passed across the viable epidermis.

The microneedle patch devices provided herein may be self-administered or administered by another individual (e.g., a parent, guardian, minimally trained healthcare worker, expertly trained healthcare worker, and/or others).

Thus, embodiments provided herein further include a simple and effective method of administering immunogenic particles with a microneedle patch device. The methods provided herein may include identifying an application site and, preferably, sanitizing the area prior to application of the drug delivery device (e.g., using an alcohol wipe). The microneedle patch device then is applied to the patient's skin/tissue and manually pressed into the patient's skin/tissue (e.g., using the thumb or finger) by applying a force as described herein.

After administration is complete, the substrate, supporting layer, housing, and/or depressible portion may be removed from the patient's skin/tissue in embodiments having separable microneedles. In one embodiment, the microneedle patch devices are used to deliver the immunogenic particles into skin by inserting the microneedles across the stratum comeum (outer 10 to 20 microns of skin that is the barrier to transdermal transport) and into the viable epidermis and dermis. The small size of the microneedles enables them to cause little to no pain and target the intradermal space. The intradermal space is highly vascularized and rich in immune cells and provides an attractive path to administer both vaccines and therapeutics. The microneedles are preferably dissolvable and once in the intradermal space they dissolve within the interstitial fluid and release the active into the skin. In embodiments that include separable microneedles, the substrate can be removed and discarded upon or after separation of the microneedles, which preferably is nearly immediately upon insertion.

In one embodiment, a method is provided for administering immunogenic particles to a patient, which includes providing one of the microneedle arrays described herein; and applying the microneedles of the array to a tissue surface of the patient, wherein the insertion of the microneedles of the array into the skin is done manually without the use of a separate or intrinsic applicator device. In this particular context, the term “applicator device” is a mechanical device that provides its own force, e.g., via a spring action or the like, which serves as the primary force to drive the microneedle array against the tissue surface, separate from any force the user may impart in holding the device and/or microneedles against the tissue surface.

As used herein, “layer” means a thickness increment, e.g., on a template for film formation, following an adsorption step. “Multilayer” means multiple (i.e., two or more) thickness increments. A “polyelectrolyte multilayer film” is a film comprising one or more thickness increments of polyelectrolytes. After deposition, the layers of a multilayer film may not remain as discrete layers. In fact, it is possible that there is significant intermingling of species, particularly at the interfaces of the thickness increments. Intermingling, or absence thereof, can be monitored by analytical techniques such as potential measurements, X-ray photoelectron spectroscopy, and time-of-flight secondary ion mass spectrometry.

“Amino acid” means a building block of a polypeptide. As used herein, “amino acid” includes the 20 common naturally occurring L-amino acids, all other natural amino acids, all non-natural amino acids, and all amino acid mimics, e.g., peptoids.

“Naturally occurring amino acids” means glycine plus the 20 common naturally occurring L-amino acids, that is, alanine, valine, leucine, isoleucine, serine, threonine, cysteine, methionine, aspartic acid, asparagine, glutamic acid, glutamine, arginine, lysine, histidine, phenylalanine, omithine, tyrosine, tryptophan, and proline.

“Non-natural amino acid” means an amino acid other than any of the 20 common naturally occurring L-amino acids. A non-natural amino acid can have either L- or D-stereochemistry.

“Amino acid sequence” and “sequence” mean a contiguous length of polypeptide chain that is at least two amino acid residues long.

“Residue” means an amino acid in a polymer or oligomer; it is the residue of the amino acid monomer from which the polymer was formed. Polypeptide synthesis involves dehydration, that is, a single water molecule is “lost” on addition of the amino acid to a polypeptide chain.

As used herein “peptide” and “polypeptide” all refer to a series of amino acids connected one to the other by peptide bonds between the alpha-amino and alpha-carboxy groups of adjacent amino acids, and may contain or be free of modifications such as glycosylation, side chain oxidation, or phosphorylation, provided such modifications, or lack thereof, do not destroy immunogenicity. As used herein, the term “peptide” is meant to refer to both a peptide and a polypeptide or protein.

A “capsule” is a polyelectrolyte film in the form of a hollow shell or a coating surrounding a core. The core comprises a variety of different encapsulants, for example, a protein, a drug, or a combination thereof. Capsules with diameters less than about 1 μm are referred to as nanocapsules. Capsules with diameters greater than about 1 μm are referred to as microcapsules.

“Cross linking” means the formation of a covalent bond, or several bonds, or many bonds between two or more molecules.

“Bioactive molecule” means a molecule, macromolecule, or macromolecular assembly having a biological effect. The specific biological effect can be measured in a suitable assay and normalizing per unit weight or per molecule of the bioactive molecule. A bioactive molecule can be encapsulated, retained behind, or encapsulated within a polyelectrolyte film. Nonlimiting examples of a bioactive molecule are a drug, a crystal of a drug, a protein, a functional fragment of a protein, a complex of proteins, a lipoprotein, an oligopeptide, an oligonucleotide, a nucleic acid, a ribosome, an active therapeutic agent, a phospholipid, a polysaccharide, a lipopolysaccharide. As used herein, “bioactive molecule” further encompasses biologically active structures, such as, for example, a functional membrane fragment, a membrane structure, a virus, a pathogen, a cell, an aggregate of cells, and an organelle. Examples of a protein that can be encapsulated or retained behind a polypeptide film are hemoglobin; enzymes, such as for example glucose oxidase, urease, lysozyme and the like; extracellular matrix proteins, for example, fibronectin, laminin, vitronectin and collagen; and an antibody. Examples of a cell that can be encapsulated or retained behind a polyelectrolyte film are a transplanted islet cell, a eukaryotic cell, a bacterial cell, a plant cell, and a yeast cell.

“Biocompatible” means causing no substantial adverse health effect upon oral ingestion, topical application, transdermal application, subcutaneous injection, intramuscular injection, inhalation, implantation, or intravenous injection. For example, biocompatible films include those that do not cause a substantial immune response when in contact with the immune system of, for example, a human being.

“Immune response” means the response of the cellular or humoral immune system to the presence of a substance anywhere in the body. An immune response can be characterized in a number of ways, for example, by an increase in the bloodstream of the number of antibodies that recognize a certain antigen. Antibodies are proteins secreted by B cells, and an immunogen is an entity that elicits an immune response. The human body fights infection and inhibits reinfection by increasing the number of antibodies in the bloodstream and elsewhere.

“Antigen” means a foreign substance that elicits an immune response (e.g., the production of specific antibody molecules) when introduced into the tissues of a susceptible vertebrate organism. An antigen contains one or more epitopes. The antigen may be a pure substance, a mixture of substances (including cells or cell fragments). The term antigen includes a suitable antigenic determinant, auto-antigen, self-antigen, cross-reacting antigen, alloantigen, tolerogen, allergen, hapten, and immunogen, or parts thereof, and combinations thereof, and these terms are used interchangeably. Antigens are generally of high molecular weight and commonly are polypeptides. Antigens that elicit strong immune responses are said to be strongly immunogenic. The site on an antigen to which a complementary antibody may specifically bind is called an epitope or antigenic determinant.

“Antigenic” refers to the ability of a composition to give rise to antibodies specific to the composition or to give rise to a cell-mediated immune response.

As used herein, a “vaccine composition” is a composition which elicits an immune response in a mammal to which it is administered, and which protects the immunized organism against subsequent challenge by the immunizing agent or an immunologically cross-reactive agent. Protection can be complete or partial with regard to reduction in symptoms or infection as compared with a non-vaccinated organism. An immunologically cross-reactive agent can be, for example, the whole protein (e.g., glucosyltransferase) from which a subunit peptide has been derived for use as the immunogen. Alternatively, an immunologically cross-reactive agent can be a different protein, which is recognized in whole or in part by antibodies elicited by the immunizing agent.

As used herein, an “immunogenic composition” is intended to encompass a composition that elicits an immune response in an organism to which it is administered, and which may or may not protect the immunized mammal against subsequent challenge with the immunizing agent. In one embodiment, an immunogenic composition is a vaccine composition.

The invention is further illustrated by the following non-limiting examples.

### EXAMPLES

### Materials and Methods

Core and Microparticle: The substrate CaCO3 cores were formed in a controlled co-precipitation reaction with the sodium salt of poly-L-glutamic acid (PGA-Na). The solutions of sodium carbonate and calcium chloride (both containing PGA-Na) were pumped through tubing and were rapidly mixed at 1:1 ratio in a flow reaction. PGA-Na provided a more stable particle and also served as the initial layering step on the CaCO3 microparticle.

The precipitated CaCO3 cores were subjected to electrostatic layer-by-layer (LbL) assembly, in which charged polymers with high net positive or net negative charges were assembled on the surface of CaCO3 microparticles. The assembly is driven by the electrostatic attraction between the soluble polymer and the oppositely charged surface. Poly-1-lysine (PLL, positive charge) and poly-1-glutamic acid (PGA, negative charge) homopolymers were alternately layered to assemble a total of 7 layers on the CaCO3 microparticle, with the 7th layer being PGA to yield a net negative surface charge. The 8th layer is the designed peptide containing a C-terminal poly-lysine tail (K20 (SEQ ID NO: 9) or K20Y (SEQ ID NO: 10)) that is net positively charged and will electrostatically layer on the negatively charged surface of the microparticle.

Homopolymers: Both PLL and PGA are commercially sourced. They are synthetically made amino acid chains that are either positively charged (PLL) or negatively charged (PGA).

Designed Peptides: Designed peptides were linearly synthesized by solid phase peptide synthesis (SPPS), a process with repeating cycles of alternating N-terminal deprotection and coupling reactions (C-terminus to N-terminus amino acid addition). The SPPS uses N-terminal FMOC protecting groups for coupling and an onium based chemistry with microwave assisted synthesis. The synthesis method for the peptide used in ACT-1216 was modified to utilize carbodiimide based chemistry for higher microwave temperature assisted synthesis that result in faster peptide coupling times. Once the peptides were synthesized, they were subjected to trifluoroacetic acid cleavage to remove any remaining protecting groups on the peptide chain, like FMOC, and the removal of the peptide from the solid support resin on the C-terminus. After cleavage, the peptides were purified through either a C4 or C18 column and lyophilized for storage.

Microneedle patch synthesis. Microneedle patch mold. Microneedle patch molds are made by laser-drilling tapered holes into silicone (polydimethylsiloxane, PDMS) sheets. Each tapered hole, corresponding to a microneedle, is 600 μm long, 200 μm at the base and 1 μm at the tip. Microneedles will be positioned at a density of 100 microneedles per square centimeter. A total of 100 microneedles per patch are used.

Microneedle patch formulation. The immunogenic particle casting solution is formulated with 15% trehalose in water to stabilize the vaccine during drying. LbL-MPs will be provided at a concentration of 1 μg antigenic peptide (DP)/μl. This is because each microneedle mold cavity has a volume of 10 nl, so that 1 μl will fill 100 microneedle mold cavities, which means that a 100-microneedle patch will contain one dose. The patch casting solution will be formulated with 50% polyvinyl alcohol and 50% sucrose. The polymer provides mechanical strength, and the sugar provides rapid dissolution and also helps stabilize the vaccine during storage.

Microneedle patch fabrication. To prepare microneedle patches, 10 μl of immunogenic particle casting solution containing LbL-MP vaccine will be cast onto the microneedle mold. After applying vacuum to pull the casting solution into the mold cavities, excess casting solution will be removed from the mold surface. The casting solution in the mold cavities will be allowed to dry. Then, 500 μl of patch casting solution (without immunogenic particles) will be cast onto the microneedle mold and allowed to dry in a chemical hood, thereby creating microneedles encapsulating immunogenic particles and a microneedle patch backing that does not contain vaccine.

### Example 1: Synthesis of Microneedle Patches

Microneedle patches were prepared with RSV and malaria model constructs ACT-1230 thru -1233 (Table 1). An 8-layer LBL-MP was prepared on a solid 3 μm CaCO3 core with FLL-FITC as the outer layer (Final architecture CaCo3:PGA:PLL:PGA:PLLPGA:PLL:PGA:PLL-FITC, where PGA-poly-L glutamate (5-15 kDa) and PLL=poly-L-lysine (5-15 kDa). The 7 (homopolypeptide) HP base layer particles were fabricated using the LbL-by-TFF method, See U.S. Pat. No. 9,975,066, incorporated herein by reference for a detailed description of the LbL-by-TFF method. This construct was labeled ACT-1229, and is also referred to as HP. The particles were suspended in 5% mannitol, 0.2% sodium carboxymethylcellulose (NaCMC) buffer and lyophilized. Quality control was done by amino acid analysis (AAA), microscopic inspection, and size distribution by dynamic light scattering. After confirmation of HP loading and recovery in both whole dissolved patch suspensions and harvested dissolved microneedle tips, LbL-MP designed peptide (DP) constructs were prepared with a DP outer layer based on Plasmodium falciparum T1BT* or RSV-GM2). Each DP was synthesized with and without an N-terminal Pam3Cys.

TABLE 1
QC DATA FOR CONSTRUCTS PREPARED FOR FORMULATION IN
TRANSDERMAL MICRONEEDLE PATCHES

| | | | CONCENTRATION | | | |
|---|---|---|---|---|---|---|
| | | | BY AAA | | | |
| | | CROSS | μg/ml @ 1% | | | ENDOTOXIN |
| CONSTRUCT | DP/EPITOPE | LINKED | DP | PLL | PGA | EU/μg |
| ACT-1229-01 | ACT-2017/PLL-FITC | N | — | 456 | 593 | — |
| ACT-1230-01 | ACT-2062/Malaria T1BT* | Y | 137 | 177 | 263 | 0.004 |
| ACT-1231-01 | ACT-2149/Pam3Cys T1BT* | Y | 67 | 182 | 267 | 0.003 |
| ACT-1232-01 | ACT-2182/RSV GM2 | N | 131 | 207 | 439 | 0.02 |
| ACT-1233-01 | ACT-2192/Pam3Cys GM2 | N | 161 | 211 | 436 | 0.005 |

TABLE 2
PEPTIDE SEQUENCES

| | | | SEQ |
|---|---|---|---|
| CON- | DP/ | | ID |
| STRUCT | EPITOPE | SEQUENCE | NO: |
| ACT- | ACT-2062/ | DPNANPNVDPNANP | 11 |
| 1230 | T1BT* | NVNANPNANPNANP | |
| | | EYLNKIQNSLSTEW | |
| | | SPCSVTSGNGK₂₀Y | |
| ACT- | ACT-2149/ | Pam3CysSKKKKDP | 12 |
| 1231 | Pam3Cys.T1BT* | NANPNVDPNANPNV | |
| | | NANPNANPNANPEY | |
| | | LNKIQNSLSTEWSP | |
| | | CSVTSGNGK₂₀Y | |
| ACT- | ACT-2182/ | NFVPCSICSNNPTC | 13 |
| 1232 | GM2 | WAICKRIPNKKPGK | |
| | | KTSGSESYIGSINN | |
| | | ITKQSASVASGSK₂₀ | |
| ACT- | ACT-2192/ | Pam3CysSKKKKNF | 14 |
| 1233 | Pam3Cys.GM2 | VPCSICSNNPTCWA | |
| | | ICKRIPNKKPGKKT | |
| | | SGSESYIGSINNIT | |
| | | KQSASVASGSK₂₀ | |
| ACT- | ACT-2182/ | NFVPCSICSNNPTC | 15 |
| 1211 | GM2 | WAICKRIPNKKPGK | |
| | | KTSGSESYIGSINN | |
| | | ITKQSASVASGSK₂₀ | |
| ACT- | ACT-2192/ | Pam3CysSKKKKNF | 16 |
| 1216 | Pam3Cys.GM2 | VPCSICSNNPTCWA | |
| | | ICKRIPNKKPGKKT | |
| | | SGSESYIGSINNIT | |
| | | KQSASVASGSK₂₀ | |
| ACT- | | Pam3Cys- | 17 |
| 2247 | | SKKKKDPNANPNVD | |
| | | PNANPNVNANPNAN | |
| | | PNAN | |
| | | PEYLNKIQNSLSTE | |
| | | WSPSSVTSGNGKKK | |
| | | KKKKKKKKKKKKKK | |
| | | KKK | |

Constructs ACT 1230-1233 were cast into microneedle patches. The PLL, PGA and DP were detectable in all patches and microneedles. (Data not shown)

### Example 2: RSV Microneedle Patch

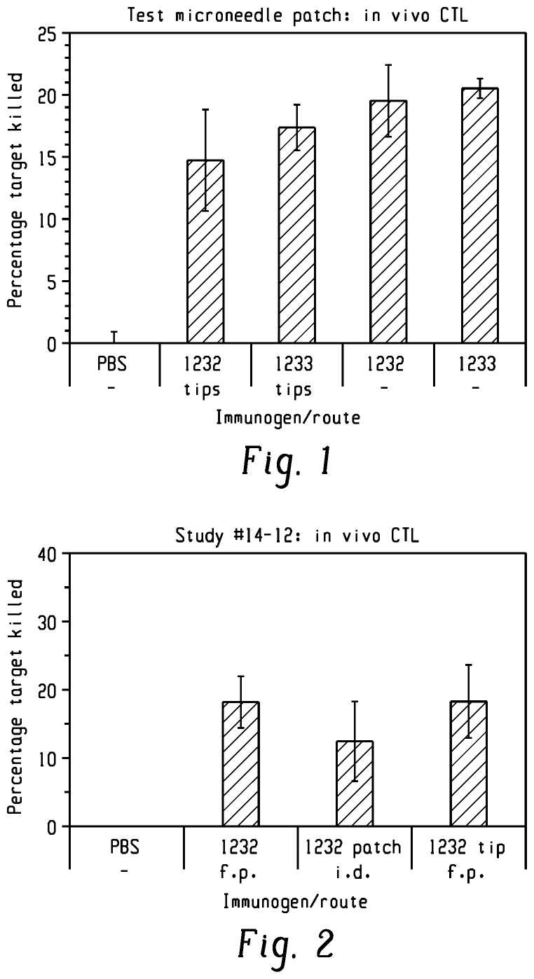

Immunological activity of recovered RSV microparticles was tested in an in vivo CTL assay. BALB/c mice were immunized via f.p. with ACT-1232 (GM2) or ACT-1233 (Pam3.GM2) LbL-MP suspensions, or with the same constructs recovered from microneedle patch tips. Mice were challenged with labeled target cells on day 7 and sacrificed the next day to measure in vivo killing of the target cells. The results in FIG. 1 show that the recovered microneedle tip suspensions induced M2-specific effector activity comparable to that induced by the control LbL-MP suspension, demonstrating that the LbL-MP retain immune potency when formulated in microneedle patches.

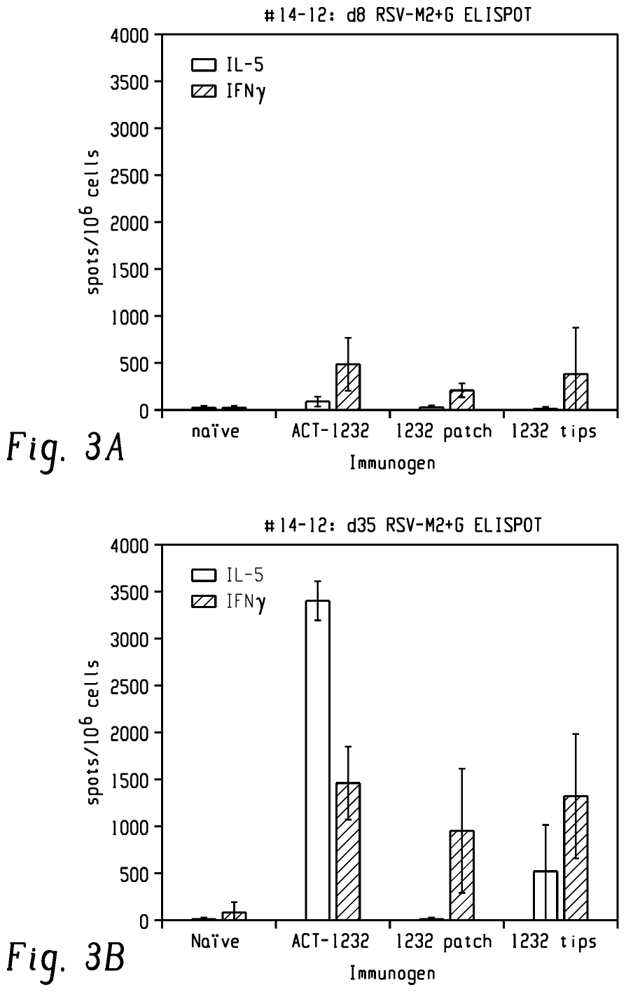

BALB/c mice were immunized by f.p. injection of ACT-1232 (positive control), application of microneedle patch loaded with ACT-1232, or f.p. injection of ACT-1232 particles recovered from the tips of loaded patches. Mice were primed on day 0 and boosted on day 28 with 1 μg DP in the positive control group (both days), and 0.6 lag for prime (day 0) and 0.9 lag for boost (day 28) for the patch and recovered tips groups. Three mice per group were challenged with M2-loaded target cells on day 7 for in vivo CTL assessment. The results in FIG. 2 show that the microneedle patch loaded with ACT-1232 induced M2-specific effector activity, albeit at levels slightly lower that those induced by the control LbL-MP or the recovered tips suspensions administered via f.p.

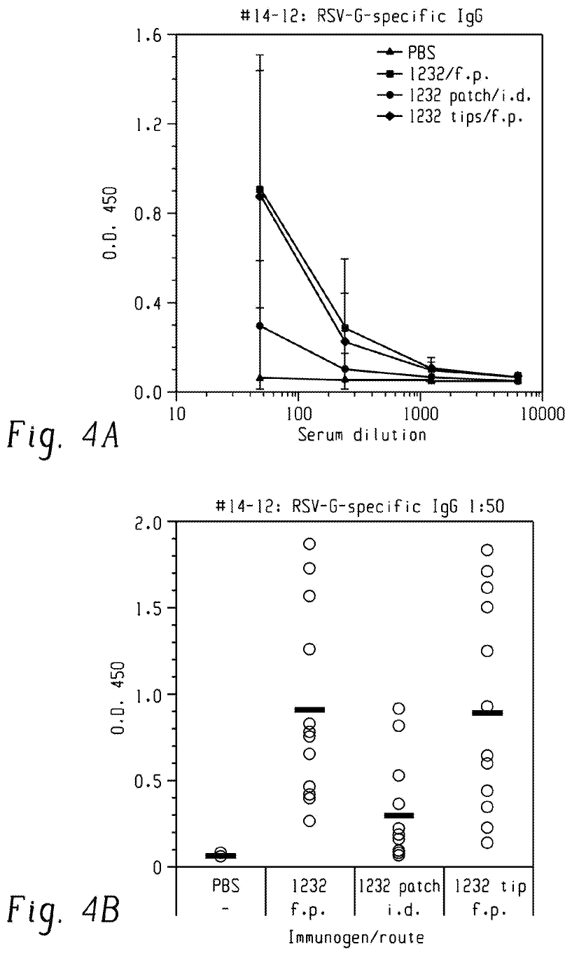

When the CTL mice were sacrificed on day 8, spleen cells were also taken for testing in B-cell and T-cell ELISPOTs. The surviving mice were bled on day 21 to assess primary antibody responses. The day 8 ELISPOT and day 21 ELISA results presented below suggested to boost all three immunized groups on day 28. On day 35, the mice were again bled to measure serum antibody titers and 3 mice per group were sacrificed for IL-5 and IFNγ ELISPOTs. The background levels in the B-cell ELISPOT were too high to yield any useful results. FIGS. 3A and B show both the day 8 and day 35 T-cell ELISPOT results. At both timepoints, all 3 immunized groups had similar IFNγ responses. The IL-5 responses on day 7 were all very low, but on day 35 there were clear differences between the groups. The ACT-1232 f.p. group had a strong IL-5 response that was completely absent in the patch group. The 1232 tip group had a low IL-5 response, which may be due to the lower dose of DP delivered at prime (0.6 lag in the tip group vs 1.0 lag for the positive control group), although the boost doses were essentially the same (0.9 lag in the tip group vs 1.0 lag for the positive control group). If the patch application was efficient, this group should have received the same amount of DP as the tip group, and we′d expect to see a similar response. Thus, the lack of IL-5 in the patch group may be due to the intradermal route of delivery compared to the footpad route in the tips group. These results suggest that intradermal microneedle delivery may favor a Th1 response with very low or no detectable Th2 response.

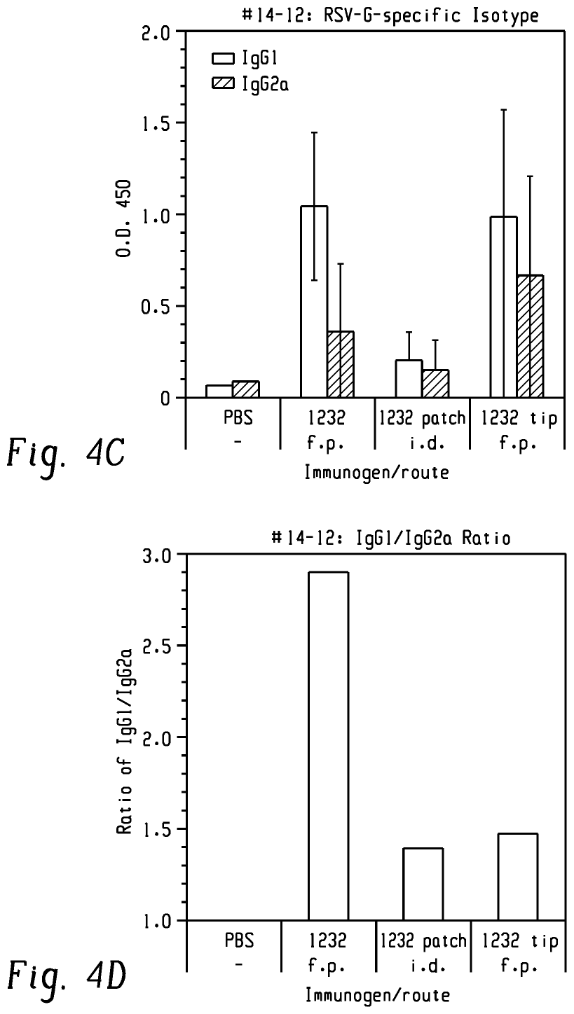

Mice were bled on day 35 (7 days post-boost) for determination of RSV-G peptide-specific antibody titers by ELISA. The results in FIGS. 4A and 4B show that microneedle patches loaded with ACT-1232 elicited lower RSV-G peptide-specific IgG responses than those induced by the control LbL-MP or the recovered tips suspensions via f. p. route. A closer examination of the antibody isotype response shows that the mice immunized by patch produced much lower levels of antigen-specific IgG1 (Th2-associated) than did the groups immunized via the f.p., with no concomitant decrease in Th1-associated IgG2a (FIG. 4C). Thus, even though the response to patch immunization is overall less potent, patch immunization appears to favor a balanced isotype response compared to f.p. immunization with native particles (FIG. 4D). This pattern of IgG1 correlates well with the IL-5 pattern seen in the T-cell ELISPOTS in FIG. 3B: both groups with measurable IL-5 ELISPOTS (Th2 cells) have IgG1 antibody responses, while the one group with no measure IL-5 has much lower levels of IgG1 antibody. It is unclear why the recovered tips administered via f.p. elicited the same levels of total IgG and IgG1 but higher levels of IgG2a than the native particles.

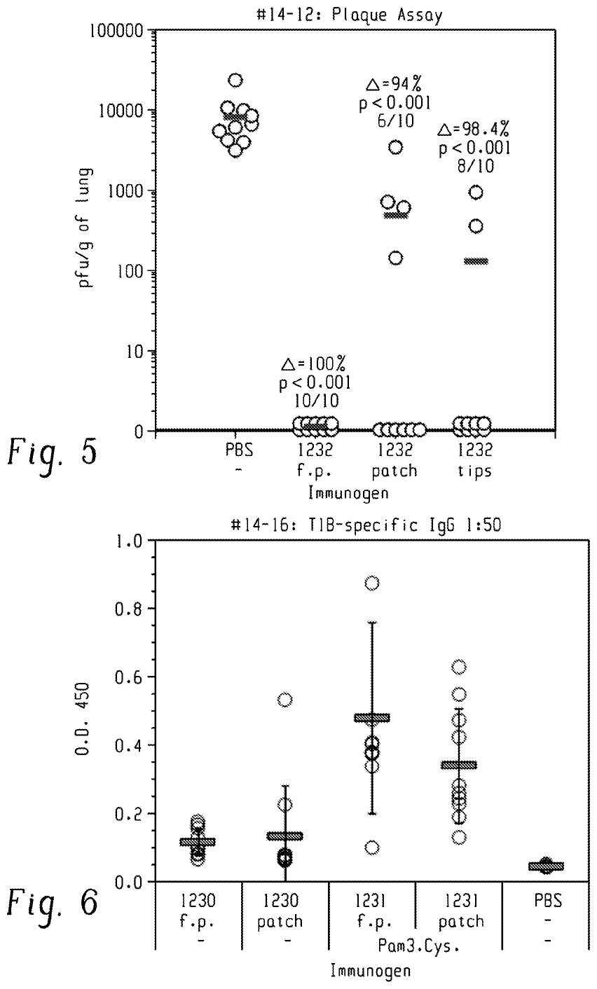

Mice (10/group) were challenged on day 50 with RSV/A2 and lungs were harvested 5 days later to assess viral burden by qPCR and plaque assay. The plaque assay results in FIG. 5 show that the ACT-1232 group (1 lag DP) was fully protected from infection, as expected. The microneedle patch and 1232 tip groups were also protected (94% and 98.4% reduction in viral burden). Although there were detectable viral titers in some of these mice, there was no statistical differences among the three immunized groups. It should be noted that the actual amount of DP delivered via patch was 0.6 lag and 0.9 lag in prime and boost, respectively, compared to 1 μg at both time points for the f.p. group. The qPCR results showed a similar trend with essentially equivalent protection in all three immunized groups (data not shown). These results demonstrate that (a) LbL-MP can be loaded in microneedle patches without losing activity, and (b) patches can deliver LbL-MP to the host and elicit a protective immune response.

### Example 3: Malaria Microneedle Patch

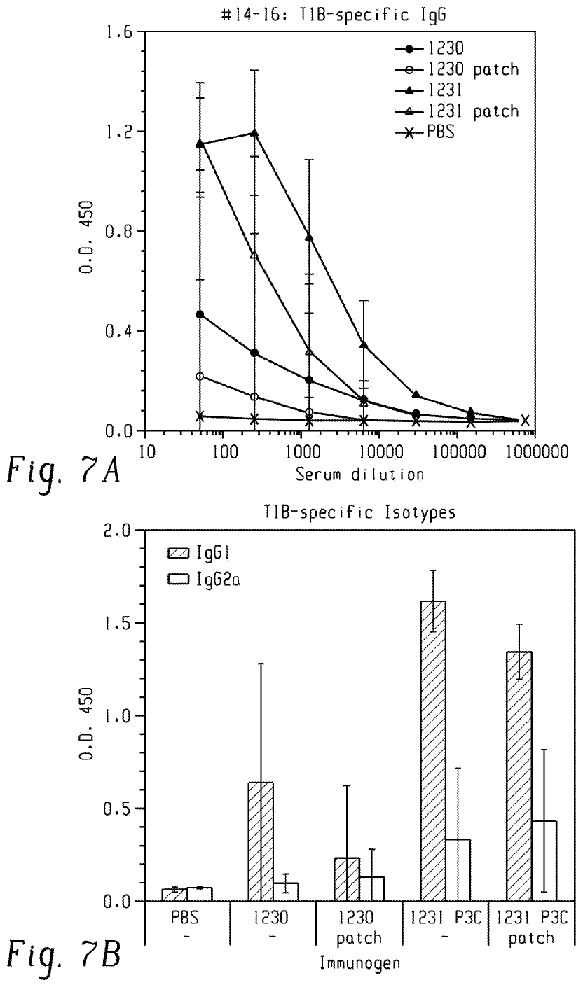

Patches containing malaria constructs ACT-1230 (T1BT*) and ACT-1231 (Pam3.Cys.T1BT*) were prepared with a target loading of 5 μg DP. C57BL/6J mice were immunized by f.p. injection of ACT-1230 or -1231 (positive control) or dermal application of microneedle patch loaded with ACT-1232 or -1231. Mice were primed on day 0 and bled on day 21 for determination of T1B-specific antibody titers by ELISA. The results in FIG. 6 show that microneedle patches loaded with ACT-1230 or -1231 elicited primary T1B-specific IgG response comparable to those induced by the control LbL-MP via f.p. route, while ACT-1231 is more potent than ACT-1230. These mice will receive a boost immunization at day 30.

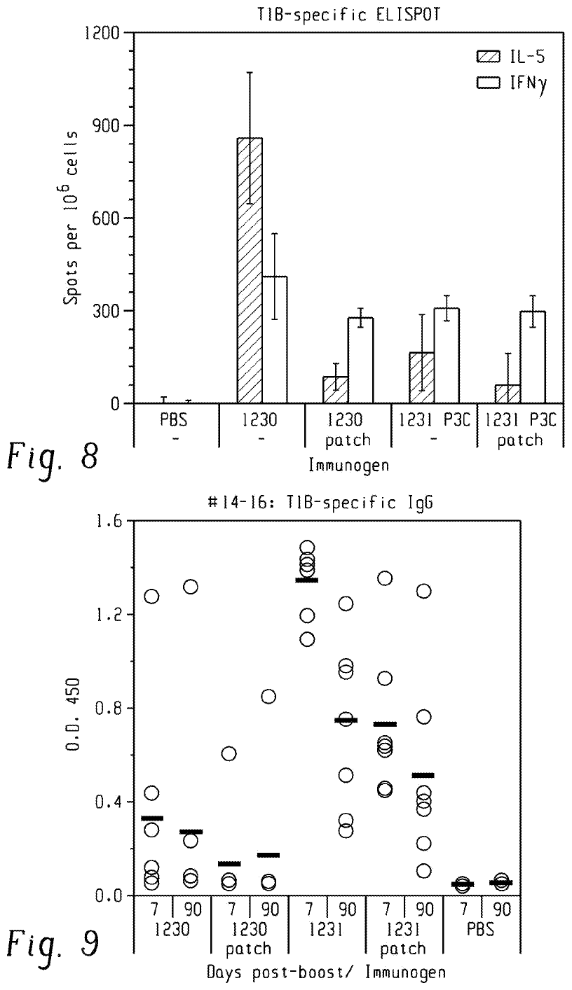

Mice immunized once by patch application developed primary T1B-specific IgG responses (FIG. 6). These mice received a boost immunization at day 30, and sera were collected on day 37 for determination of T1B-specific antibody titers by ELISA. The results in FIG. 7A show that microneedle patches loaded with ACT-1230 or -1231 elicited antibody responses to T1B peptide, albeit at levels slightly lower than those induced by the control LbL-MP, while ACT-1231 is more potent than ACT-1230. A closer examination of the antibody isotype response shows that the mice immunized by 1230 patch produced slightly lower levels of antigen-specific IgG1 (Th2-associated) than did the group immunized with 1230 via the f.p. (FIG. 7B).

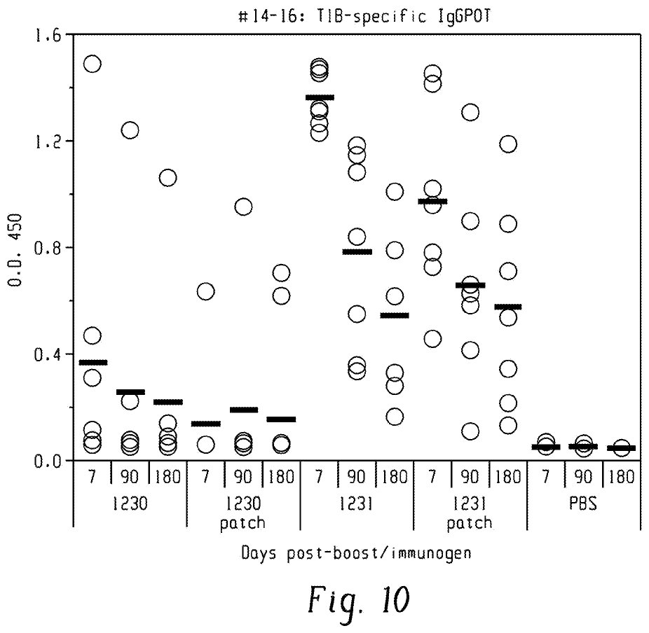

On day 37, 3 mice per group were sacrificed for IL-5 and IFNγ ELISPOTs. The results in FIG. 8 show that while all immunized groups had similar IFNγ responses, the group immunized by f.p. injection of ACT-1230 had much higher IL-5 responses than any other group. This result shows that modification of the DP with Pam3Cys (ACT-1231) reduces the IL-5 response regardless of the mode of administration, confirming our previous observations, and intradermal delivery replicated this response even in the absence of Pam3Cys. This pattern of lower Th2 response in the microneedle patch-immunized group reproduces the pattern seen in a similar study using RSV microparticles (See Example 2).

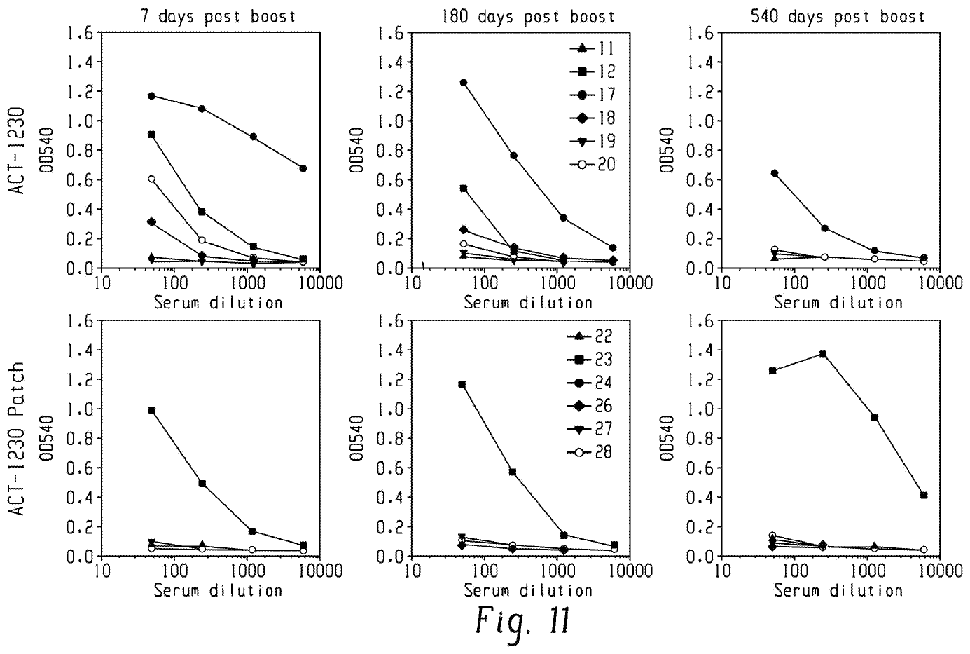

Since these mice studied above were not challenged with sporozoites, we held them on study to enable monitoring of the persistence of immunity. These mice were bled on day 120 (90 days post-boost), and sera were analyzed in T1B ELISA along with the day 37 (7 days post-boost) sera which had been stored at −20° C. The results in FIG. 9 show only a modest decrease in antibody titer from 7 to 90 days post-boost, indicating that the antibody response persists for 3 months post-boost.

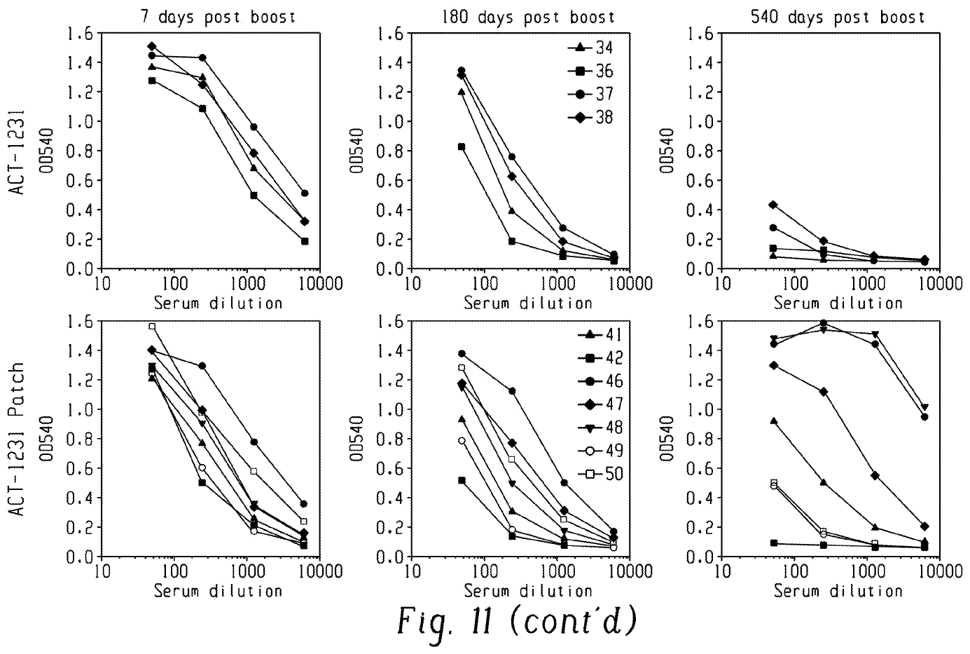

These same mice were bled on day 210 (180 days post-boost), and sera were analyzed in T1B ELISA along with the day 37 (7 days post-boost) and day 120 (90 days post-boost) sera which had been stored at −20° C. The results in FIG. 10 show only a modest decrease in antibody titer from 7 to 180 days post-boost, indicating that the antibody response persists for 6 months post-boost.

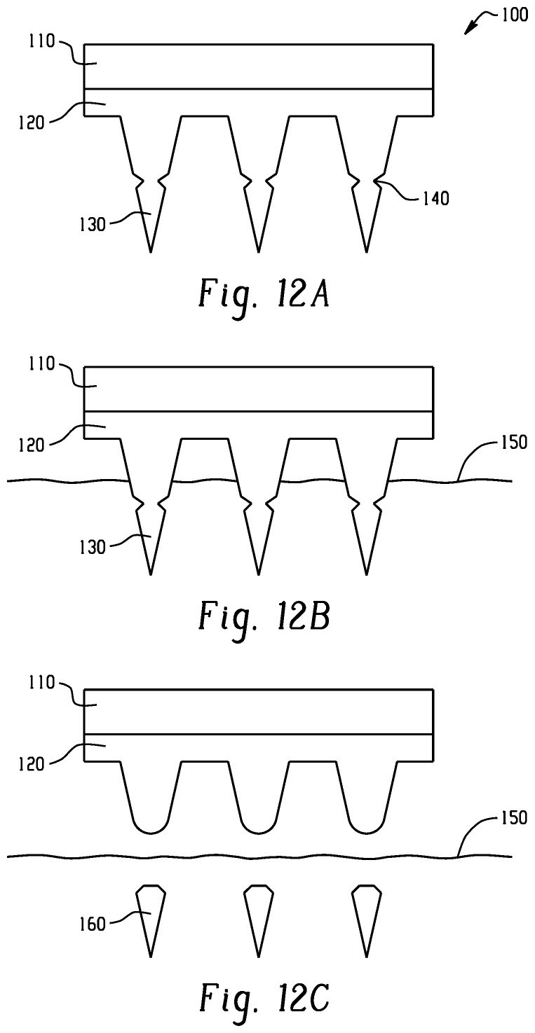

These same mice were bled on day 570 (540 days post-boost), and sera were analyzed in T1B ELISA along with the day 37 (7 days post-boost) and day 210 (180 days post-boost) sera that had been stored at −20° C. The results in FIG. 11 show that antibody levels in the patch-immunized mice remained high at day 540, compared to an overall decrease in antibody levels in mice immunized by injection. There are notable exceptions to this pattern, however. For example, mouse 23 in the ACT-1230 patch group was the only one that had a detectable response even at 7 dpb, and her antibody levels remained high at 180 dpb and even increased by 540 dpb. In the ACT-1231 patch group, all mice had robust responses at 7 dpb and there was only a slight decrease in most mice by 180 dpb, the exception being mouse 46 that did not experience a decreased response by 180 dpb. The most surprising data are in the ACT-1231 patch 540 dpb group, where three of the mice have higher responses than at 180 dpb (46 green, 47 purple, and 48 light blue). It is not known why these individual mice experience a long-term increase in circulating antibody levels, but it is clear that this phenotype is specific to intradermal administration of LbL-MP by microneedle patches.

### Example 4: Comparison of Microneedle Patch to IM Injection

Microparticles (LbL-MP) were fabricated by alternately layering poly-1-glutamic acid (PGA, negative charge) and poly-1-lysine (PLL, positive charge) on CaCO3 cores with addition of DP (ACT-2247) as the outermost layer. The particles are referred to as ACT-1250, CaCO₃ with PGA/PLL/PGA/PLL/PGA/PLL-FITC/PGA/ACT-2247.

DP loading was analyzed by amino acid analysis (AAA) and size distribution was analyzed by dynamic light scattering (DLS). Lyophilized particles were resuspended in a polyvinyl alcohol/sucrose solution and MN patches were prepared using this particle dispersion by casting onto MN molds. MNPs (microneedle patches) were applied to BALB/c mice for dose determination and prime vs. prime-boost application, and comparison with IM injection for immune response.

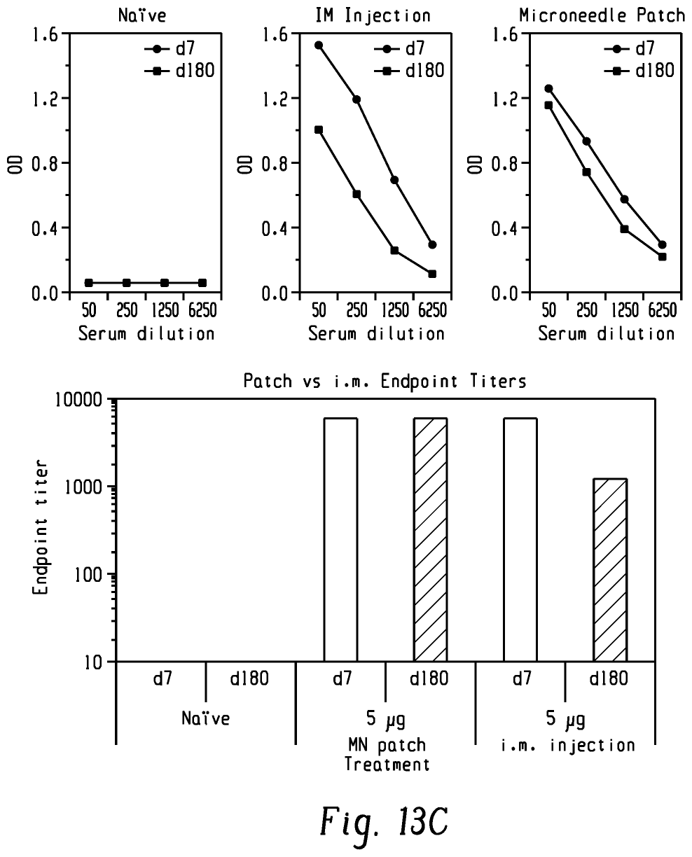

Vaccine-loaded LbL-MP were successfully cast into MNPs, retaining their particle size and integrity. MNPs were prepared with 0.2, 1 and 5 μg DP. All patches showed full insertion into mouse skin. Immunization with MN patches elicited T1B-specific IgG responses comparable to those induced by the same doses of the same vaccine delivered via IM route (FIG. 13a). The strongest immune response was achieved with 5 μg DP without signs of inflammation. Continued studies with this dose. MN patch immunization elicited more potent IFNγ (Th1) responses even after the prime dose and much higher levels after the boost dose compared to the IM groups (FIG. 2b). Antibody responses in the MNP group persisted at day 180 post-boost with minimal drop compared to 7 days post-boost, while responses to the same vaccine injected IM dropped precipitously by day 180 post-boost (FIG. 2C).

MN patches with sufficient strength for skin insertion were fabricated with vaccine-loaded LbL-MPs, maintaining the MP properties. LbL-MPs were delivered intradermally to mice by MN patch. MN patch immunization elicited immune responses in the absence of overt signs of inflammation and favored a Th1 phenotype antibody response. The immunized host maintained a long-lived antibody response comparable to that elicited by IM immunization.

The use of the terms “a” and “an” and “the” and similar referents (especially in the context of the following claims) are to be construed to cover both the singular and the plural, unless otherwise indicated herein or clearly contradicted by context. The terms first, second, etc., as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of eliciting an immune response in human subject in need thereof, comprising
transdermally delivering to the human subject a composition comprising immunogenic particles by a microneedle patch device, wherein transdermally delivering comprises:
transdermally delivering an initial dose by the microneedle patch device;
optionally transdermally delivering a booster dose by the microneedle patch device within 3-12 weeks of the initial dose; and
transdermally delivering a subsequent dose by the microneedle patch device at 1 year or more after delivering the initial dose or the optional booster dose, wherein no dose is given between the optional booster dose and the subsequent dose;
wherein the composition comprising immunogenic particles comprises
a multilayer film, the multilayer film comprising two or more layers of charged polyelectrolytes, wherein adjacent layers comprise oppositely charged polyelectrolytes, one of the charged polyelectrolyte layers in the multilayer film comprises an antigenic polyelectrolyte comprising a peptide epitope covalently linked to the antigenic polyelectrolyte,
wherein the polyelectrolytes that are not the antigenic polyelectrolyte comprise a polycationic material or a polyanionic material having a molecular weight of greater than 1,000 and at least 5 charges per molecule, and
wherein the multilayer film is deposited on a core particle or forms a hollow particle to provide the composition; and
wherein the microneedle patch device comprises a substrate comprising an array of microneedles extending therefrom, wherein the microneedles comprise the composition comprising immunogenic particles.

2. The method of claim 1, wherein the microneedles are bioerodible microneedles.

3. The method of claim 1, wherein the microneedles comprise a matrix material, such as polyvinyl alcohol, dextran, carboxymethylcellulose, maltodextrin, sucrose, trehalose, or a combination thereof.

4. The method of claim 1, comprising delivering the subsequent dose at more than two years after delivering the booster dose.

5. The method of claim 1, comprising delivering the subsequent dose at more than three years after delivering the booster dose.

6. The method of claim 1, wherein the microneedles have a height between 100 μm and 2 mm.

7. The method of claim 1, wherein the microneedles are solid microneedles.

8. The method of claim 1, wherein the antigenic polyelectrolyte is an antigenic polypeptide.

9. The method of claim 1, wherein the antigenic polyelectrolyte further comprises a covalently linked TLR ligand.

10. The method of claim 1, wherein the antigenic polyelectrolyte is in the outermost later of the multilayer film.

11. The method of claim 1, wherein the peptide epitope is a viral, bacterial, fungal, or parasite epitope.

12. The method of claim 1, wherein the peptide epitope is a Plasmodium falciparum circumsporozoite protein T1, B, T8 epitope, or a combination thereof.

13. The method of claim 1, wherein the peptide epitope comprises a modified Plasmodium falciparum T* epitope of SEQ ID NO: 4 or SEQ ID NO: 5.

14. The method of claim 1, wherein the peptide epitope is an RSV-G, RSV-F, RSV-M2 epitope, or a combination thereof.

15. The method of claim 1, wherein the microneedle patch device comprises a housing having a depressible portion; a substrate having a microneedle side and an opposing back side; the array of microneedles extending from the microneedle side of the substrate, wherein the microneedles comprise the immunogenic particles; and a supporting layer arranged on the opposing back side of the substrate, and movably mounted within the housing; wherein the depressible portion is configured to apply or activate upon depression a shearing force to at least one of the supporting layer and substrate effective to separate the array of microneedles from the substrate.

16. The method of claim 1, wherein one or more of the microneedles of the array of microneedles comprises at least one feature configured to separate the one or more microneedles from the substrate upon application of the shearing force.

17. The method of claim 15, wherein the at least one feature comprises a predefined fracture region at a proximal end of one or more microneedles of the array of microneedles and/or the substrate located about each of the one or more microneedles of the array of microneedles.


SEQUENCE LISTING (17 sequences)

SEQ ID NO: 1 - protein, 16 aa, Plasmodium falciparum
DPNANPNVDPNANPNV

SEQ ID NO: 2 - protein, 4 aa, Plasmodium falciparum
NANP

SEQ ID NO: 3 - protein, 20 aa, Plasmodium falciparum
EYLNKIQNSLSTEWSPCSVT

SEQ ID NO: 4 - protein, 20 aa, synthetic construct
  source 1..20: note = Modified T* epitope
EYLNKIQNSLSTEWSPSSVT

SEQ ID NO: 5 - protein, 20 aa, synthetic construct
  source 1..20: note = Modified T* epitope
EYLNKIQNSLSTEWSPASVT

SEQ ID NO: 6 - protein, 298 aa, Respiratory syncytial virus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 7 - protein, 574 aa, Respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 8 - protein, 192 aa, Respiratory syncytial virus
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTDTNDHAKNNDTT

SEQ ID NO: 9 - protein, 20 aa, synthetic construct
  source 1..20: note = Poly-lysine tail
KKKKKKKKKKKKKKKKKKKK

SEQ ID NO: 10 - protein, 21 aa, synthetic construct
  source 1..21: note = Poly-lysine tail
KKKKKKKKKKKKKKKKKKKKY

SEQ ID NO: 11 - protein, 73 aa, synthetic construct
  source 1..73: note = Designed polypeptide ACT-1230
DPNANPNVDPNANPNVNANPNANPNANPEYLNKIQNSLSTEWSPCSVTSGNGKKKKKKKKKKKKKKKKKKKKY

SEQ ID NO: 12 - protein, 78 aa, synthetic construct
  source 1..78: note = Designed polypeptide ACT-1231
  LIPID 1: note = Pam3Cys
SKKKKDPNANPNVDPNANPNVNANPNANPNANPEYLNKIQNSLSTEWSPCSVTSGNGKKKKKKKKKKKKKKKKKKKKY

SEQ ID NO: 13 - protein, 74 aa, synthetic construct
  source 1..74: note = Designed polypeptide ACT-1232
NFVPCSICSNNPTCWAICKRIPNKKPGKKTSGSESYIGSINNITKQSASVASGSKKKKKKKKKKKKKKKKKKKK

SEQ ID NO: 14 - protein, 79 aa, synthetic construct
  source 1..79: note = Designed polypeptide ACT-1233
  LIPID 1: note = Pam3Cys
SKKKKNFVPCSICSNNPTCWAICKRIPNKKPGKKTSGSESYIGSINNITKQSASVASGSKKKKKKKKKKKKKKKKKKKK

SEQ ID NO: 15 - protein, 74 aa, synthetic construct; identical to SEQ ID NO: 13
  source 1..74: note = Designed polypeptide ACT-1211

SEQ ID NO: 16 - protein, 79 aa, synthetic construct; identical to SEQ ID NO: 14
  source 1..79: note = Designed polypeptide ACT-1216
  LIPID 1: note = Pam3Cys

SEQ ID NO: 17 - protein, 77 aa, synthetic construct
  source 1..77: note = Designed polypeptide ACT-2247
  LIPID 1: note = Pam3Cys
SKKKKDPNANPNVDPNANPNVNANPNANPNANPEYLNKIQNSLSTEWSPSSVTSGNGKKKKKKKKKKKKKKKKKKKK